(19) United States
(12) Patent Application Publication
(10) Pub. No.: US 2024/0245426 A1
(43) Pub. Date: Jul. 25, 2024

(54) CIRCUMCISION EQUIPMENT

(71) Applicant: Wuhu ShangRing Technology Co., Ltd, Wuhu (CN)

(72) Inventors: Shujie Xia, Shanghai (CN); Huarong Yu, Beijing (CN); Jingjing Shang, Wuhu (CN); Jianzhong Shang, Wuhu (CN)

(21) Appl. No.: 18/624,091

(22) Filed: Apr. 1, 2024

Related U.S. Application Data
(62) Division of application No. 16/531,139, filed on Aug. 5, 2019, now Pat. No. 11,980,390.
Application No. 16/531,139 is a continuation of application No. PCT/CN2018/074988, filed on Feb. 1, 2018.

(30) Foreign Application Priority Data
Feb. 4, 2017 (CN) 201710063339.X
Feb. 4, 2017 (CN) 201710063340.2
Feb. 4, 2017 (CN) 201710065136.4
Feb. 4, 2017 (CN) 201710065137.9
Feb. 4, 2017 (CN) 201710065138.3
Feb. 4, 2017 (CN) 201710065139.8
Feb. 4, 2017 (CN) 201710065140.0
Feb. 4, 2017 (CN) 201710065146.8
Feb. 4, 2017 (CN) 201710065147.2
Feb. 4, 2017 (CN) 201710065148.7
Feb. 4, 2017 (CN) 201710065149.1
Feb. 4, 2017 (CN) 201710065150.4
Feb. 4, 2017 (CN) 201720109034.3
Apr. 18, 2017 (CN) 201710250780.9
Apr. 18, 2017 (CN) 201710250951.8
Apr. 19, 2017 (CN) 201710254989.2
Apr. 19, 2017 (CN) 201710255021.1
Apr. 19, 2017 (CN) 201710255023.0
Apr. 19, 2017 (CN) 201710255025.X
Apr. 24, 2017 (CN) 201710273894.5
Apr. 24, 2017 (CN) 201710273895.X
Apr. 24, 2017 (CN) 201710274306.X
Apr. 24, 2017 (CN) 201710274307.4
Apr. 24, 2017 (CN) 201710274308.9
Apr. 24, 2017 (CN) 201710274309.3
Apr. 24, 2017 (CN) 201710274396.2
Apr. 24, 2017 (CN) 201710274397.7
Apr. 24, 2017 (CN) 201710274398.1
Apr. 24, 2017 (CN) 201710274399.6
Apr. 24, 2017 (CN) 201710274400.5
Apr. 24, 2017 (CN) 201710274426.X
Aug. 18, 2017 (CN) 201710709597.0

(51) Int. Cl. A61B 17/326; A61B 17/00; A61B 17/068; A61B 17/072; A61B 90/00

(52) U.S. Cl. CPC A61B 17/326; A61B 17/0682; A61B 17/072; A61B 90/08; A61B 2017/00561; A61B 2017/07228; A61B 2017/07285; A61B 2090/0811

(57) ABSTRACT

The present invention relates to surgical devices, particularly to a circumcision apparatus, enable the circumcision apparatus to be suitable for different surgery objects and environments.

### CROSS REFERENCE TO RELATED APPLICATIONS

The present application is a Divisional Application of U.S. patent application Ser. No. 16/531,139 filed on Aug. 5, 2019, which is a Continuation Application of PCT Application No. PCT/CN2018/074988 filed on Feb. 1, 2018, which claims the benefit of Chinese Patents Application Ser. Nos. 20710063339.X, 201710065146.8, 201710065140.0, 201710065139.8, 201710065138.3, 201710065150.4, 201710065149.1, 201710065148.7, 201710065147.2, 201710063340.2, 201710065137.9, 201710065136.4, 201720109034.3 filed on Feb. 4, 2017, Chinese Patents Application Ser. Nos. 201710250780.9 and 201710250951.8 filed on Apr. 18, 2017, Chinese Patents Application Ser. Nos. 201710254989.2, 201710255021.1, 201710255023.0 and 201710255025.X filed on Apr. 19, 2017, Chinese Patent Application Nos. 201710274426.X, 201710274400.5, 201710274399.6, 201710274398.1, 201710274397.7, 201710274396.2, 201710274309.3, 201710274308.9, 201710274307.4, 201710274306.X, 201710273895.X and 201710273894.5 filed on Apr. 24, 2017, and Chinese Patent Application Ser. No. 201710709597.0 filed on Aug. 18, 2017. All the above are hereby incorporated by reference.

### FIELD OF THE INVENTION

The present invention relates to surgical devices, particularly to a circumcision apparatus and more particularly to a blood vessel docking apparatus of a built-in or valgus circumcision device; a built-in or valgus graduated circumcision device; a prepuce circumcision stapler with an indication device, which is, more particularly, of a built-in type and a valgus type; an operation-visible circumcision stapler.

### BACKGROUND OF THE INVENTION

Refer to the blood vessel docking apparatus of a built-in or valgus circumcision device. The redundant prepuce or phimosis is one of the causes of male urinary system infections and sexually transmitted diseases. The redundant prepuce or phimosis can cause urinary tract infections, resulting in chronic prostatitis, with a series of symptoms, such as pains in back and waist, impotence and premature ejaculation. Therefore, the removal of the redundant prepuce is a good way to prevent these diseases.

Traditionally, surgical removal of the phimosis or redundant prepuce has the main technical points of removal of excess prepuce, hemostasis, and apposition suture of cut edges to skin. A postoperative patient cannot move around, suffers unbearable pain in each change of ointment and endures huge pain when stitches are taken out finally. In addition, incomplete ligating hemostasis will cause prepuce hematoma, thus requiring a surgical treatment again. Furthermore, since the prepuce removal and the hemostasis process are performed separately, the surgery time is prolonged, and the patient's panic is exacerbated.

A therapeutic method of applying a laser and high-frequency electric surgical knife technology to circumcision has been developed at present. Although this therapeutic method substitutes for scissors cutting and makes a bleeding spot coagulated, the patient's tissues will be burnt and susceptible to infection.

For the built-in type, a circumcision device in which a prepuce incision is sutured using suturing nails at one time was developed in this field. However, in the prior art, a circumcision cover only cooperates with a comprehensive cover and the circumcision and U-nail hemostasis are performed after clamping is completed. Since the position of the nail groove disposed in the comprehensive cover is always invisible, the prepuce blood vessel cannot be accurately positioned, and the U-nail in the U-nail groove cannot accurately correspond to the prepuce blood vessel during surgery. Thus, the surgical hemostasis effect is not good. Even the nail head of the U-nail may be right hammered into the blood vessel, and surgery risk is caused as blood cannot be punctually stopped.

For the valgus type, a circumcision device in which a prepuce incision is sutured using suturing nails at one time was developed in this field. However, in the prior art, a valgus circumcision device only adopts an upper cover and a lower cover cooperating with each other and an inner ring and an inner comprehensive cover cooperating with each other, and the circumcision and U-nail hemostasis are performed after clamping is completed. Since the position of the internally disposed nail groove is always invisible, the prepuce blood vessel cannot be accurately positioned, and the U-nail in the U-nail groove cannot accurately correspond to the prepuce blood vessel during surgery. Thus, the surgical hemostasis effect is not good, even the nail head of the U-nail may be right hammered into the blood vessel, and surgery risk is caused as blood cannot be punctually stopped.

Refer to the built-in or valgus graduated circumcision device. The redundant prepuce or phimosis is one of the causes of male urinary system infections and sexually transmitted diseases. The redundant prepuce or phimosis can cause urinary tract infections, resulting in chronic prostatitis, with a series of symptoms, such as pains in back and waist, impotence and premature ejaculation. Therefore, the removal of the redundant prepuce is a good way to prevent these diseases.

Traditionally, surgical removal of the phimosis or redundant prepuce has the main technical points of removal of excess prepuce, hemostasis, and apposition suture of cut edges to skin. A postoperative patient cannot move around, suffers unbearable pain in each change of ointment and endures huge pain when stitches are taken out finally. In addition, incomplete ligating hemostasis will cause prepuce hematoma, thus requiring a surgical treatment again. Furthermore, since the prepuce removal and the hemostasis processes are performed separately, the surgery time is prolonged, and the patient's panic is exacerbated.

A therapeutic method of applying a laser and high-frequency electric surgical knife technology to circumcision has been developed at present. Although this therapeutic method substitutes for scissors cutting and makes a bleeding spot coagulated, the patient's tissues will be burnt and susceptible to infection.

For the built-in type circumcision device, a prepuce incision is sutured using suturing nails at one time. However, in the prior art, the positions of nail grooves arranged in a comprehensive cover will be invisible once the cooperation of the comprehensive cover and a circumcision cover is completed. Therefore, U-nails in the U-nail grooves cannot accurately correspond to prepuce blood vessels during surgery, resulting in a poor surgical hemostasis effect. Even the nail heads of the U-nails may be just hammered into the blood vessels, resulting in a surgery risk as bleeding cannot be punctually stopped.

For the valgus circumcision device, a prepuce incision is sutured using suturing nails at one time. However, in the prior art, the positions of nail grooves of an inner comprehensive cover in a lower cover will be invisible once the cooperation of an upper cover and the lower cover is completed. Therefore, U-nails in the U-nail grooves cannot accurately correspond to prepuce blood vessels during surgery, resulting in a poor surgical hemostasis effect. Even the nail heads of the U-nails may be just hammered into the blood vessels, resulting in a surgery risk as bleeding cannot be punctually stopped.

Refer to the prepuce circumcision stapler with an indication device, which is, more particularly, of a built-in type and a valgus type.

The redundant prepuce or phimosis is one of the causes of male urinary system infections and sexually transmitted diseases. The redundant prepuce or phimosis can cause urinary tract infections, resulting in chronic prostatitis, with a series of symptoms, such as pains in back and waist, impotence and premature ejaculation. Therefore, the removal of the redundant prepuce is a good way to prevent these diseases.

Traditionally, surgical removal of the phimosis or redundant prepuce has the main technical points of removal of excess prepuce, hemostasis, and apposition suture of cut edges to skin. A postoperative patient cannot move around, suffers unbearable pain in each change of ointment and endures huge pain when stitches are taken out finally. In addition, incomplete ligating hemostasis will cause prepuce hematoma, thus requiring a surgical treatment again. Furthermore, since the prepuce removal and the hemostasis processes are performed separately, the surgery time is prolonged, and the patient's panic is exacerbated.

A therapeutic method of applying a laser and high-frequency electric surgical knife technology to circumcision has been developed at present. Although this therapeutic method substitutes for scissors cutting and makes a bleeding spot coagulated, the patient's tissues will be burnt and susceptible to infection.

Then, a prepuce circumcision device was developed in this field. For the built-in type prepuce circumcision device, a prepuce incision is sutured using suturing nails at one time. However, in the prior art, the positions of nail grooves arranged in a comprehensive cover will be invisible once the cooperation of the comprehensive cover and a prepuce circumcision cover is completed. Therefore, U-nails in the U-nail grooves cannot accurately correspond to prepuce blood vessels during surgery, resulting in a poor surgical hemostasis effect. Even the nail heads of the U-nails may be just hammered into the blood vessels, resulting in a surgery risk as bleeding cannot be punctually stopped.

For the valgus prepuce circumcision device, a prepuce incision is sutured using suturing nails at one time. However, in the prior art, the positions of nail grooves of an inner comprehensive cover in a lower cover will be invisible once the configuration of the inner ring and the cooperation of an upper cover and the lower cover is completed. Therefore, U-nails in the U-nail grooves cannot accurately correspond to prepuce blood vessels during surgery, resulting in a poor surgical hemostasis effect. Even the nail heads of the U-nails may be just hammered into the blood vessels, resulting in a surgery risk as bleeding cannot be punctually stopped.

Refer to the operation-visible circumcision stapler. The redundant prepuce or phimosis is one of the causes of male urinary system infections and sexually transmitted diseases. The redundant prepuce or phimosis can cause urinary tract infections, resulting in chronic prostatitis, with a series of symptoms, such as pains in back and waist, impotence and premature ejaculation. Therefore, the removal of the redundant prepuce is a good way to prevent these diseases.

Traditionally, surgical removal of the phimosis or redundant prepuce has the main technical points of removal of excess prepuce, hemostasis, and apposition suture of cut edges to skin. A postoperative patient cannot move around, suffers unbearable pain in each change of ointment and endures huge pain when stitches are taken out finally. In addition, incomplete ligating hemostasis will cause prepuce hematoma, thus requiring a surgical treatment again. Furthermore, since the prepuce removal and the hemostasis processes are performed separately, the surgery time is prolonged, and the patient's panic is exacerbated.

A therapeutic method of applying a laser and high-frequency electric surgical knife technology to circumcision has been developed at present. Although this therapeutic method substitutes for scissors cutting and makes a bleeding spot coagulated, the patient's tissues will be burnt and susceptible to infection.

Then, a prepuce circumcision device was developed in this field. A prepuce incision is sutured using suturing nails at one time. However, in the prior art, the positions of nail grooves arranged in a comprehensive cover will be invisible once the cooperation of the comprehensive cover and a prepuce circumcision cover is completed. Therefore, U-nails in the U-nail grooves cannot accurately correspond to prepuce blood vessels during surgery, resulting in a poor surgical hemostasis effect. Even the nail heads of the U-nails may be just hammered into the blood vessels, resulting in a surgery risk as bleeding cannot be punctually stopped.

### SUMMARY OF THE INVENTION

Refer to the blood vessel docking apparatus of a built-in or valgus circumcision device.

Regarding the existing technical problems, the purpose of the present invention is to provide a blood vessel docking apparatus of a built-in circumcision device, which can accurately determine the corresponding situation of the prepuce blood vessel and the U-nail when the circumcision cover cooperates with the comprehensive cover through the blood vessel corresponding/docking devices arranged on the circumcision cover and the comprehensive cover, so as to perform final positioning of the circumcision cover and the comprehensive cover after the blood vessel is accurately positioned, thereby greatly improving the effect of surgical hemostasis.

The purpose of the present invention is to further provide a blood vessel docking apparatus of a valgus circumcision device, which can accurately determine the corresponding situation of the prepuce blood vessel and the U-nail when the upper cover cooperates with the lower cover and the inner ring cooperates with the inner comprehensive cover through the blood vessel docking device arranged on the inner ring, so as to perform final positioning of the inner ring, the inner comprehensive cover, the upper cover and the lower cover after the blood vessel is accurately positioned, thereby greatly improving the effect of surgical hemostasis.

The specific technical solution is as follows:

A blood vessel docking apparatus for a circumcision device comprises a circumcision cover and a comprehensive cover, and further comprises blood vessel corresponding grooves and/or blood vessel docking grooves, wherein the circumcision cover and the comprehensive cover are configured to be assembled or connected with each other; a plurality of blood vessel corresponding grooves is disposed along a periphery of a front end of the circumcision cover; and/or a plurality of blood vessel docking grooves is disposed along a periphery of a front end of the comprehensive cover.

Further, front ends of the blood vessel corresponding grooves are close to an annular top end of the circumcision cover, and back ends of the blood vessel corresponding grooves are away from the annular top end of the circumcision cover backwards relative to the front ends of the blood vessel corresponding grooves.

Further, the blood vessel corresponding grooves obliquely extend backwards from the front end of the circumcision cover.

Further, a top end of the comprehensive cover is provided with an annular outer wall, the blood vessel docking grooves are disposed on an outer surface of the annular outer wall, front ends of the blood vessel docking grooves are flush with a top end of the annular outer wall, or the front ends of the blood vessel docking grooves are higher than the top end of the annular outer wall, or the front ends of the blood vessel docking grooves are lower than the top end of the annular outer wall.

Further, back ends of the blood vessel docking grooves are away from the top end of the annular outer wall backwards relative to the front ends of the blood vessel docking grooves.

Further, the blood vessel docking grooves obliquely extend backwards from the top end of annular outer wall.

Further, the comprehensive cover is provided with a plurality of U-nail grooves for receiving U-nails and nail outlets of the U-nail grooves are disposed on or close to the top end of the comprehensive cover.

Further, the nail outlets of the U-nail grooves are disposed in the annular outer wall.

Further, the plurality of blood vessel corresponding grooves correspond to the blood vessel docking grooves, and/or the U-nail grooves, and/or the U-nails in the U-nail grooves one by one; and/or, the plurality of blood vessel docking grooves correspond to the blood vessel corresponding grooves, and/or the U-nail grooves, and/or the U-nails in the U-nail grooves one by one; and/or, the width of the plurality of blood vessel corresponding grooves corresponds to the width of the blood vessel docking grooves, and/or the width of the U-nail grooves, and/or the width of the U-nails in the U-nail grooves; and/or, the width of the plurality of blood vessel docking grooves corresponds to the width of the blood vessel corresponding grooves, and/or the width of the U-nail grooves, and/or the width of the U-nails in the U-nail grooves.

Further, an arc-shaped transition surface is arranged between the blood vessel corresponding grooves and the annular top end of the circumcision cover; and/or, an arc-shaped transition surface is arranged between the blood vessel corresponding grooves and convex plates formed among the blood vessel docking grooves; and/or, the front ends of the blood vessel docking grooves are narrower than the back ends thereof; and/or, a U-nail top ring and a lower comprehensive cover are further included, the back end of the circumcision cover is provided with a positioning rod matched and connected with a positioning groove of the U-nail top ring, and a back end of the comprehensive cover is connected to a lower comprehensive cover.

A blood vessel docking apparatus for a valgus circumcision device comprises an inner ring, an inner comprehensive cover and blood vessel docking grooves, wherein the inner ring and the inner comprehensive cover are configured to be assembled or connected with each other; and a plurality of blood vessel docking grooves is disposed along a periphery of a back end of the inner ring.

Further, the inner comprehensive cover is provided with a plurality of U-nail grooves for receiving U-nails and the U-nail grooves are disposed along a periphery of a front end of the inner comprehensive cover.

Further, the nail outlets of the U-nail grooves are disposed on or close to the top end of the inner comprehensive cover.

Further, the positions of the plurality of blood vessel docking grooves correspond to the positions of the U-nail grooves, and/or the positions of the U-nails in the U-nail grooves one by one; and/or, the width of the plurality of blood vessel docking grooves correspond to the width of the U-nail grooves, and/or the width of the U-nails in the U-nail grooves.

Further, the blood vessel docking grooves are preset on the inner ring, or the blood vessel docking grooves are prefabricated rings and capable of sleeving the inner ring.

Further, back ends of the blood vessel docking grooves are close to an annular back end of the inner ring or flush with the back end of the inner ring, and front ends of the blood vessel docking grooves are away from the annular back end of the inner ring forwards relative to the back ends of the blood vessel docking grooves.

Further, the apparatus comprises a U-nail top ring to be assembled and matched with the inner comprehensive cover to push the U-nail out of the U-nail groove.

Further, the apparatus comprises an upper cover and a lower cover, wherein the upper cover and the lower are assembled and connected with each other, the inner ring is arranged in the upper cover, and all or part of the inner comprehensive cover is disposed in the lower cover.

Further, the apparatus comprises a comprehensive cover, wherein a front end of the comprehensive cover is connected to a back end of the lower cover, and all or part of the U-nail top ring is disposed in the comprehensive cover.

For the built-in type, the present invention can accurately determine the corresponding situation of the prepuce blood vessel and the U-nails when the circumcision cover cooperates with the comprehensive cover through the blood vessel corresponding/docking devices arranged on the circumcision cover and the comprehensive cover, so as to perform final positioning of the circumcision cover and the comprehensive cover after the blood vessel is accurately positioned, thereby greatly improving the effect of surgical hemostasis. More preferably, the blood vessel corresponding grooves and the blood vessel docking grooves can be flexibly arranged so as to be in one-to-one correspondence with the U-nail grooves, the U-nails in the U-nail grooves, the intervals among the U-nail grooves, and the cooperating relationship between the blood vessel corresponding grooves and the blood vessel docking grooves, the widths of the blood vessel corresponding grooves and the blood vessel docking grooves and so on, thereby meeting the requirements of different circumcision devices and different surgeries. The U-nail main surface closes the blood vessels, so that hemostasis is good during surgery, and necrosis is accelerated after surgery.

For the valgus type, compared with the prior art, the present invention can accurately determine the corresponding situation of the prepuce blood vessel and the U-nails when the upper cover cooperates with the lower cover and the inner ring cooperates with the inner comprehensive cover through the blood vessel docking devices arranged on the inner ring, so as to perform final positioning of the inner ring, the inner comprehensive cover, the upper cover and the lower cover after the blood vessel is accurately positioned, thereby greatly improving the effect of surgical hemostasis. More preferably, the blood vessel docking grooves can be flexibly arranged so as to be in one-to-one correspondence with the U-nail grooves, the U-nails in the U-nail grooves, cooperation relationship of the intervals among the U-nail grooves, and the widths of the blood vessel corresponding grooves and the blood vessel docking grooves and so on, thereby meeting the requirements of different circumcision devices and different surgeries. The U-nail main surface closes the blood vessels, so that hemostasis is good during surgery, and necrosis is accelerated after surgery.

Refer to the built-in or valgus graduated circumcision device.

Regarding the above technical problems, an object of the present invention is to provide a built-in graduated circumcision device which can accurately determine the corresponding situation of prepuce blood vessels and U-nails by means of a graduation device arranged on the comprehensive cover, thereby greatly improving the effect of surgical hemostasis.

Regarding the above technical problems, an object of the present invention is to further provide a valgus graduated circumcision device which can accurately determine the corresponding situation of prepuce blood vessels and U-nails by means of a graduation device arranged on an upper cover, a lower cover or a locking ring, thereby greatly improving the effect of surgical hemostasis.

The specific technical solutions are as below.

A built-in graduated circumcision device comprises a comprehensive cover, a circumcision cover and a graduation device, wherein the comprehensive cover and the circumcision cover are assembled to and cooperate with each other; and the graduation device is arranged on the comprehensive cover.

Further, the graduation device is arranged at the end, close to the circumcision cover, of an outer surface of the comprehensive cover.

Further, the graduation device comprises a ring of graduations annularly arranged on a periphery of the end, close to the circumcision cover, of the comprehensive cover.

Further, a plurality of U-nail grooves which constitutes a ring is arranged in the comprehensive cover.

Further, graduation marks on the graduation device are arranged to be in one-to-one correspondence with the U-nail grooves and/or intervals among the U-nail grooves and/or U-nails in the U-nail grooves.

Further, the graduation marks on the graduation device are arranged such that the width between the two graduation marks is equal to the width of the U-nail groove and/or the width of the U-nail in the U-nail groove and/or the width of the interval between the U-nail grooves.

Further, the graduation device is graduations carved on the comprehensive cover in advance and/or a graduation label adhered to the comprehensive cover and/or a prefabricated graduation ring or graduation strip sleeving the comprehensive cover.

Further, the circumcision device is of a flat head or oblique head type.

Further, a ring of blood vessel docking grooves are annularly arranged on the outer surface of the comprehensive cover, and the graduation marks on the graduation device are arranged to be in one-to-one correspondence with the blood vessel docking grooves and/or intervals among the blood vessel docking grooves.

Further, a ring of blood vessel corresponding grooves are annularly arranged on an outer surface of the circumcision cover, and the graduation marks on the graduation device are arranged to be in one-to-one correspondence with the blood vessel corresponding grooves and/or the intervals among the blood vessel corresponding grooves.

A valgus graduated circumcision device comprises an upper cover and/or a lower cover, and/or a locking ring, and further comprises a graduation device, wherein the upper cover and the lower cover are assembled to and cooperate with each other; the locking ring is used for locking the upper cover and the lower cover; and the graduation device is arranged on the upper cover, and/or the lower cover, and/or the locking ring.

Further, the graduation device is arranged at the end, close to the lower cover, of an outer surface of the upper cover.

Further, the graduation device comprises a ring of graduations annularly arranged on a periphery of the end, close to the lower cover, of the upper cover.

Further, the graduation device is arranged at the end, close to the upper cover, of an outer surface of the lower cover.

Further, the graduation device comprises a ring of graduations annularly arranged on a periphery of the end, close to the upper cover, of the lower cover.

Further, the graduation device is arranged on a periphery of the locking ring.

The valgus graduated circumcision device further comprises an inner comprehensive cover arranged in the lower cover, wherein a plurality of U-nail grooves are annularly arranged on a periphery of the inner comprehensive cover and constitute a ring.

Further, graduation marks on the graduation device are arranged to be in one-to-one correspondence with the U-nail grooves and/or intervals among the U-nail grooves and/or U-nails in the U-nail grooves.

Further, the graduation marks on the graduation device are arranged such that the width between the two graduation marks is equal to the width of the U-nail groove and/or the width of the U-nail in the U-nail groove and/or the width of the interval between the U-nail grooves.

Further, the graduation device is graduations carved on the upper cover, and/or the lower cover, and/or the locking ring; and/or a graduation label adhered to the upper cover, and/or the lower cover, and/or the locking ring; and/or a prefabricated graduation ring or graduation strip sleeving the upper cover, and/or the lower cover, and/or the locking ring.

For the built-in type, compared with the prior art at the present, the present invention can accurately determine the corresponding situation of prepuce blood vessels and U-nails by means of the graduation device arranged on the comprehensive cover, so that the U-nails tightly ties the blood vessels to the greatest extent, thereby greatly improving the effect of surgical hemostasis. More preferably, the graduation marks on the graduation device can be flexibly arranged to be in one-to-one correspondence with the U-nail grooves, the U-nails in the U-nail grooves, the intervals among the U-nail grooves, the blood vessel docking grooves, the intervals among the blood vessel docking grooves, the blood vessel corresponding grooves, the intervals among the blood vessel corresponding grooves, etc., thereby meeting the requirements of different circumcision devices and different surgeries. The U-nail main surfaces close the blood vessels finally, so that hemostasis is good during surgery, and necrosis is accelerated after surgery.

For the valgus type, compared with the prior art at the present, the present invention can accurately determine the corresponding situation of prepuce blood vessels and U-nails by means of the graduation device arranged on the upper cover, the lower cover or the locking ring, so that the U-nails tightly ties the blood vessels to the greatest extent, thereby greatly improving the effect of surgical hemostasis. More preferably, the graduation marks on the graduation device can be flexibly arranged to be in one-to-one correspondence with the U-nail grooves, the U-nails in the U-nail grooves, the intervals among the U-nail grooves, etc., thereby meeting the requirements of different circumcision devices and different surgeries. The U-nail main surfaces close the blood vessels finally, so that hemostasis is good during surgery, and necrosis is accelerated after surgery.

Refer to the prepuce circumcision stapler with an indication device, which is, more particularly, of a built-in type and a valgus type.

Regarding the above existing technical problems, an objective of the present invention is to provide a prepuce circumcision stapler with an indication device, which is, more particularly, of a built-in type and a valgus type. For the built-in type prepuce circumcision stapler, the corresponding situation of prepuce blood vessels and U-nails can be accurately judged during surgery by means of an indication device arranged on a comprehensive cover, thereby greatly improving the effect of surgical hemostasis. For the valgus prepuce circumcision stapler, the corresponding situation of prepuce blood vessels and U-nails can be accurately judged during surgery by means of an indication device arranged on an upper cover, a lower cover, an inner ring or a locking ring, thereby greatly improving the effect of surgical hemostasis. The specific technical solution is as follows.

A built-in prepuce circumcision stapler with an indication device comprises a comprehensive cover, a prepuce circumcision cover and the indication device, wherein the comprehensive cover and the prepuce circumcision cover are assembled to and cooperate with each other; the indication device is arranged on the comprehensive cover.

Further, the indication device is arranged at one end, close to the prepuce circumcision cover, of the outer surface of the comprehensive cover.

Further, the indication device is a ring of protrusions or recesses which are annularly arranged on the periphery of one end, close to the prepuce circumcision cover, of the comprehensive cover; preferably, the concave or convex degree of the protrusions or recesses allows that the protrusions or recesses can be contacted and perceived by an operator when the prepuce covers or does not cover the protrusions or recesses.

Further, a plurality of U-nail grooves which serves to accommodate U-nails and constitutes a ring is arranged in the comprehensive cover.

Further, the protrusions or recesses of the indication device are arranged to be in one-to-one correspondence with the U-nail grooves and/or intervals among the U-nail grooves and/or the U-nails in the U-nail grooves; or, intervals among the protrusions or recesses of the indication device are in one-to-one correspondence with the U-nail grooves and/or the intervals among the U-nail grooves and/or the U-nails in the U-nail grooves.

Further, the protrusions or recesses of the indication device are arranged such that the width between the two protrusions or recesses is equal to or slightly smaller than the width of the U-nail groove, and/or the width of the U-nail in the U-nail groove, and/or the width of the interval between the U-nail grooves.

Further, the indication device is protrusions or recesses prefabricated on the comprehensive cover, and/or an indication label which is adhered to the comprehensive cover and has protrusions or recesses, and/or a prefabricated indication ring or indication strip which sleeves the comprehensive cover and has protrusions or recesses.

Further, the protrusions are convex blocks and/or convex points and/or convex strips, and the recesses are grooves and/or concave points and/or concave strips; preferably, both the left side surface and the right side surface and/or both the upper end surface and the lower end surface of the convex block have one or a plurality of oblique surfaces; and/or, one or two of the convex points constitute a group and are circular convex points; and/or one or two of the convex strips constitute a group, and both the left side surface and the right side surface of each convex strip have one or a plurality of oblique surfaces; and/or, both the left side surface and the right side surface and/or both the upper end surface and the lower end surface of the groove have one or a plurality of oblique surfaces; and/or, one or two of the concave points constitute a group and are circular concave points; and/or, one or two of the concave strips constitute a group, and both the left side surface and the right side surface of each concave strip have one or a plurality of oblique surfaces; and/or, the convex strip is a vertical convex strip, and the upper end and the lower end of the convex strip are curved; and/or, the concave strip is a vertical concave strip, and the upper end and the lower end of the convex strip are curved.

Further, the prepuce circumcision stapler is of a flat head or oblique head type.

Further, a ring of blood vessel docking grooves are annularly arranged on the outer surface of the comprehensive cover, and the protrusions or recesses on the indication device are arranged to be in one-to-one correspondence with the blood vessel docking grooves and/or intervals among the blood vessel docking grooves; and/or a ring of blood vessel corresponding grooves are annularly arranged on the outer surface of the prepuce circumcision cover, and the protrusions or recesses on the indication device are arranged to be in one-to-one correspondence with the blood vessel corresponding grooves and/or intervals among the blood vessel corresponding grooves.

A valgus prepuce circumcision stapler with an indication device comprises an upper comprehensive cover, a lower comprehensive cover and/or a connecting cover, a prepuce fixing hoop, a U-nail device and the indication device, wherein the upper comprehensive cover and the lower comprehensive cover are assembled to and cooperate with each other; or, the lower part of the upper comprehensive cover is connected to the connecting cover, and the lower part of the connecting cover is connected to the lower comprehensive cover; the prepuce fixing hoop is arranged in the upper comprehensive cover, or the prepuce fixing hoop and the upper comprehensive cover are of an integral structure; the U-nail device is fully or partially arranged in the lower comprehensive cover; or the U-nail device is fully or partially arranged in the connecting cover; or, a part of the U-nail device is arranged in the lower comprehensive cover, and the other part of the U-nail device is arranged in the connecting cover; the indication device is arranged on the upper comprehensive, and/or the lower comprehensive cover, and/or the prepuce fixing hoop, and/or the U-nail device.

Further, the valgus prepuce circumcision stapler with the indication device further comprises a locking ring which is used for locking the upper comprehensive cover, and/or the connecting cover, and/or the lower comprehensive cover and is provided with the indication device.

Further, the indication device is a ring of protrusions or recesses which are annularly arranged; preferably, the concave or convex degree of the protrusions or recesses allows that the protrusions or recesses can be contacted and perceived by an operator when the prepuce covers or does not cover the protrusions or recesses.

Further, the U-nail device comprises a U-nail top ring and a thimble guide block; the thimble guide block is used for accommodating suturing U-nails, and/or guiding the movement of the suturing U-nails, and/or positioning and/or guiding a thimble of the U-nail top ring; the U-nail top ring is used for pushing the U-nails in the thimble guide block out in cooperation with the thimble guide block; the indication device is arranged on the thimble guide block.

Further, the thimble guide block is provided with a plurality of thimble guide grooves which are annularly arranged on the annular thimble guide block and are capable of accommodating the suturing U-nails.

Further, the protrusions or recesses of the indication device are arranged to be in one-to-one correspondence with the thimble guide grooves and/or intervals among the thimble guide grooves and/or U-nails in the thimble guide grooves; or, intervals among the protrusions or recesses of the indication device are in one-to-one correspondence with the thimble guide grooves and/or the intervals among the thimble guide grooves and/or U-nails in the thimble guide grooves.

Further, the protrusions or recesses of the indication device are arranged such that the width between the two protrusions or recesses is equal to or slightly smaller than the width of the thimble guide groove, and/or the width of the U-nail in the thimble guide groove, and/or the width of the interval between the thimble guide grooves.

Further, the indication device is protrusions or recesses which are prefabricated on the upper comprehensive cover, and/or the lower comprehensive cover, and/or the prepuce fixing hoop, and/or the U-nail device, and/or the locking ring; and/or, the indication device is an indication label which is adhered to the upper comprehensive cover, and/or the lower comprehensive cover, and/or the prepuce fixing hoop, and/or the U-nail device, and/or the locking ring and has protrusions or recesses; and/or, the indication device is a prefabricated indication ring or indication strip which sleeves the upper comprehensive cover, and/or the lower comprehensive cover, and/or the prepuce fixing hoop, and/or the U-nail device, and/or the locking ring and has protrusions or recesses; and/or, the indication device is protrusions or recesses which are prefabricated on the periphery of the upper end and/or the periphery of the lower end of the prepuce fixing hoop, and/or an indication label which is adhered to the periphery of the upper end and/or the periphery of the lower end of the prepuce fixing hoop and has protrusions or recesses, and/or a prefabricated indication ring or strip which sleeves the periphery of the upper end and/or the periphery of the lower end of the prepuce fixing hoop and has protrusions or recesses; and/or, the indication device is protrusions or recesses which are prefabricated on the periphery of the upper end and/or the periphery of the lower end of the thimble guide block, and/or an indication label which is adhered to the periphery of the upper end and/or the periphery of the lower end of the thimble guide block and has protrusions or recesses, and/or a prefabricated indication ring or strip which sleeves the periphery of the upper end and/or the periphery of the lower end of the thimble guide block and has protrusions or recesses; and/or, the indication device is graduations prefabricated on the periphery of the upper end and/or the periphery of the lower end of the prepuce fixing hoop, and/or a graduation label adhered to the periphery of the upper end and/or the periphery of the lower end of the prepuce fixing hoop, and/or a prefabricated graduation ring or graduation strip which sleeves the periphery of the upper end and/or the periphery of the lower end of the prepuce fixing hoop, and/or a blood vessel docking groove which is prefabricated on the periphery of the upper end and/or the periphery of the lower end of the prepuce fixing hoop; and/or, the indication device is graduations prefabricated on the periphery of the upper end and/or the periphery of the lower end of the U-nail device, and/or a graduation label adhered to the periphery of the upper end and/or the periphery of the lower end of the U-nail device, and/or a prefabricated graduation ring or graduation strip which sleeves the periphery of the upper end and/or the periphery of the lower end of the U-nail device, and/or a blood vessel docking groove which is prefabricated on the periphery of the upper end and/or the periphery of the lower end of the U-nail device.

Further, the protrusions are convex blocks and/or convex points and/or convex strips, and the recesses are grooves and/or concave points and/or concave strips.

Further, both the left side surface and the right side surface and/or both the upper end surface and the lower end surface of the convex block have one or a plurality of oblique surface; and/or, one or two of the convex points constitute a group and are circular convex points; and/or one or two of the convex strips constitute a group, and both the left side surface and the right side surface of each convex strip have one or a plurality of oblique surfaces; and/or, both the left side surface and the right side surface and/or both the upper end surface and the lower end surface of the groove have one or a plurality of oblique surfaces; and/or, one or two of the concave points constitute a group and are circular concave points; and/or, one or two of the concave strips constitute a group, and both the left side surface and the right side surface of each concave strip have one or a plurality of oblique surfaces; and/or, the convex strip is a vertical convex strip, and the upper end and the lower end of the convex strip are curved; and/or, the concave strip is a vertical concave strip, and the upper end and the lower end of the convex strip are curved.

Further, the indication device is arranged at one end, close to the upper comprehensive cover, of the outer surface of the lower comprehensive cover; and/or, the indication device is arranged at one end, close to the connecting cover, of the outer surface of the lower comprehensive cover; and/or, the indication device is arranged at one end, close to the lower comprehensive cover or the connecting cover, of the outer surface of the upper comprehensive cover; and/or, the indication device is arranged at the upper end of the outer surface of the upper comprehensive cover; and/or, the indication device is arranged at one end, close to the upper comprehensive cover, of the outer surface of the connecting cover; and/or, the indication device is arranged at one end, close to the lower comprehensive cover, of the outer surface of the connecting cover; and/or, the indication device is arranged at the upper end of the outer surface of the prepuce fixing hoop; and/or, the indication device is arranged at the lower end of the outer surface of the prepuce fixing hoop; and/or, the indication device is arranged at the upper end of the outer surface of the U-nail device; and/or, the indication device is arranged at the lower end of the outer surface of the U-nail device; and/or, the indication device is annularly arranged on the periphery of the upper part/the lower part of the thimble guide block.

For the built-in type, compared with the prior art at the present, the present invention can accurately determine the corresponding situation of prepuce blood vessels and U-nails by means of the indication device arranged on the comprehensive cover, so that the U-nails tightly tie the blood vessels to the greatest extent, thereby greatly improving the effect of surgical hemostasis. More preferably, the concave or convex structure on the indication device can be flexibly arranged to be in one-to-one correspondence with the U-nail grooves, the U-nails in the U-nail grooves, the intervals among the U-nail grooves, the blood vessel docking grooves, the intervals among the blood vessel docking grooves, the blood vessel corresponding grooves, the intervals among the blood vessel corresponding grooves, etc., thereby meeting the requirements of different circumcision devices and different surgeries. The U-nail main surfaces close the blood vessels finally between the U-nails and the blood vessels, so that hemostasis is good during surgery, and necrosis is accelerated after surgery.

For the valgus type, compared with the prior art at the present, the present invention can accurately determine the corresponding situation of prepuce blood vessels and U-nails by means of the indication device arranged on the upper cover, the lower cover, the inner ring or the locking ring, so that the U-nails tightly tie the blood vessels to the greatest extent, thereby greatly improving the effect of surgical hemostasis. More preferably, the concave or convex structure of the indication device can be flexibly arranged to be in one-to-one correspondence with the U-nail grooves, the U-nails in the U-nail grooves, the intervals among the U-nail grooves, etc., thereby meeting the requirements of different circumcision devices and different surgeries. The U-nail main surfaces close the blood vessels finally between the U-nails and the blood vessels, so that hemostasis is good during surgery, and necrosis is accelerated after surgery.

Refer to the operation-visible circumcisionstapler. Regarding the above problems in the prior art, an object of the present invention is to provide an operation-visible circumcision stapler. In the operation-visible circumcision stapler, by arranging the comprehensive cover, the U-nail top ring and other cooperation structures to be transparent and visible, the corresponding situation of prepuce blood vessels and U-nails may be determined accurately during a surgery, and thus the surgical hemostasis effect is improved greatly. The specific technical solution is as follows.

Provided is an operation-visible circumcision stapler which comprises a comprehensive cover and a circumcision cover, wherein the comprehensive cover and the circumcision cover are assembled to and cooperate with each other; and an upper portion, close to the circumcision cover, of the comprehensive cover or the entire comprehensive cover is transparent or semi-transparent.

Further, a plurality of U-nail grooves which is used for accommodating U-nails and constitutes a ring is arranged in the comprehensive cover.

Further, there are intervals among the U-nail grooves; the inner surfaces and/or outer surfaces of the U-nail grooves are made of a transparent or semi-transparent material; and the intervals are made of a transparent or semi-transparent material.

Further, the operation-visible circumcision stapler further comprises a U-nail top ring which is fully or partially embedded into the comprehensive cover, and thimbles in one-to-one correspondence with the U-nail grooves are arranged at the top end of the U-nail top ring.

Further, the thimble of the U-nail top ring or the entire U-nail top ring is transparent or semi-transparent.

Further, the operation-visible circumcision stapler further comprises a circumcision knife which is partially or fully embedded into the U-nail top ring.

Further, the circumcision cover has a lower end inserted into the comprehensive cover and an upper end supported at the top end of the comprehensive cover for cooperating with the comprehensive cover to clamp prepuce.

Further, blood vessel corresponding grooves are annularly arranged at the upper end of the circumcision cover and are in one-to-one correspondence with the U-nail grooves.

Further, a positioning rod is arranged at the lower end of the circumcision cover; a positioning hole is formed in the U-nail top ring; and the positioning rod is configured to be inserted in and be positioned in the positioning hole, so that after the circumcision cover is positioned, the blood vessel corresponding grooves on the circumcision cover are in one-to-one correspondence with the U-nail grooves.

Further, a ring of blood vessel docking grooves/graduations is annularly arranged on the outer surface of the comprehensive cover, and the blood vessel docking grooves/graduations are in one-to-one correspondence with the U-nail grooves and/or the blood vessel corresponding grooves.

Compared with the prior art at present, the present invention has the advantages that by arranging the comprehensive cover, the U-nail top ring and other cooperation structures to be transparent and visible, the corresponding situation of prepuce blood vessels and U-nails may be determined accurately during a surgery, and thus the U-nails tightly tie the blood vessels to the greatest extent, thereby greatly improving the surgical hemostasis effect. More preferably, the blood vessel corresponding grooves arranged on the circumcision cover are in one-to-one correspondence with the U-nail grooves, the U-nails in the U-nail grooves, the intervals among the U-nail grooves, the blood vessel docking grooves, the intervals among the blood vessel docking grooves, the blood vessel corresponding grooves, the intervals among the blood vessel corresponding grooves and the like, and the blood vessels are positioned through the operator's vision and touch, so that demands of different circumcision devices and different surgery are met and finally main surfaces of the U-nails close the blood vessels between the U-nails and the blood vessels. Thus, an excellent hemostasis effect during the surgery is achieved and the postoperative necrosis process is accelerated.

### BRIEF DESCRIPTION OF THE DRAWINGS

Refer to the blood vessel docking apparatus of a built-in or valgus circumcision device.

FIG. 1 is an exploded view of a built-in flat-head circumcision device arranged with a blood vessel docking apparatus in the built-in type;

FIG. 2 is a partial enlarged view of the circumcision cover in the built-in type;

FIG. 3 is a partial enlarged view of U-nail top ring in the built-in type;

FIG. 4 is a partial enlarged view of the comprehensive cover in the built-in type;

FIG. 5 is a partial enlarged view of the U-nails in the built-in type;

FIG. 6 is a partial enlarged view of the U-nail grooves in the built-in type;

FIG. 7 is a partial enlarged view of the lower end of the circumcision cover in the built-in type;

FIG. 8 is an overall view of the built-in flat-head circumcision device arranged with a blood vessel docking apparatus in the built-in type;

FIG. 9 is an exploded view of a built-in oblique-head circumcision device arranged with a blood vessel docking apparatus in the built-in type;

FIG. 10 is a partial enlarged view of the circumcision cover in the built-in type;

FIG. 11 is a partial enlarged view of the U-nail top ring in the built-in type;

FIG. 12 is a partial enlarged view of the comprehensive cover in the built-in type;

FIG. 13 is a partial enlarged view of the U-nail grooves in the built-in type;

FIG. 14 is a partial enlarged view of the lower end of the circumcision cover in the built-in type;

FIG. 15 is an overall view of the built-in oblique-head circumcision device arranged with a blood vessel docking apparatus in the built-in type; this figures includes: circumcision cover 1, blood vessel corresponding groove 1-1, negative pressure hole 1-2, positioning rod 1-3, positioning groove 1-3-1, U-nail seat 1-4, circumcision knife washer 2, U-nail backing ring 3, circumcision knife 4, U-nail 5, comprehensive cover 6, U-nail groove 6-1, blood vessel docking groove 6-2, U-nail top ring 7, U-nail top column 7-1, positioning hole 7-2, positioning groove 7-2-1, lower comprehensive cover 8, connecting block 9, closing mark 9-1, opening mark 9-2, opening guide groove 9-3, positioning convex point 9-4, pin hole 9-5, pin hole 9-6, pin 10-1, pin 10-2, third pin 10-3, fourth pin 10-4, fifth pin 10-5, sixth pin 10-6, connecting rod 11, screw threads 11-1, screw threads 11-2, small connecting rod 12, pin hole 12-1, pin hole 12-2, small connecting rod 13, pin hole 13-1, pin hole 13-2, handle 14, pin hole 14-1, pin hole 14-2, handle 15, pin hole 15-1, pin hole 15-2, opening knob 16, pointer 16-1, sliding block 16-2, spring 17, sliding rod 18, pin hole 18-1, pin hole 18-2, knob 19, connecting screw threads 19-1, positioning concave point 19-2;

FIG. 16 is an exploded view of a valgus circumcision device arranged with a blood vessel positioning apparatus in the valgus type;

FIG. 17 is a partial enlarged view of the inner ring in the valgus type;

FIG. 18 is a partial enlarged view of the upper cover in the valgus type; this figure includes: plug 1, positioning concave point 1-1, sliding rod 2, spring supporting platform 2-1, pin hole 2-2, pin hole 2-3, hook 2-4, spring 3, opening knob 4, pointer 4-1, sliding block 4-2, connecting rod 5, small connecting rod 5-1, pin hole 5-1-1, pin hole 5-1-2, small connecting rod 5-2, pin hole 5-2-1, pin hole 5-2-2, pin 6-1, pin 6-2, pin 6-3, pin 6-4, pin 6-5, pin 6-6, handle 7-1, pin hole 7-1-1, pin hole 7-1-2, handle 7-2, pin hole 7-2-1, pin hole 7-2-2, connecting block 8, clamping platform 8-1, pin hole 8-2, opening guide groove 8-3, closing mark 8-4, opening mark 8-5, hook 8-6, pin hole 8-7, positioning convex point 8-8, comprehensive cover 9, clamping groove 9-1, U-nail top ring 10, groove 10-1, U-nail top column 10-2, lower cover 11, clamping platform 11-1, positioning platform 11-2, U-nail 12, circumcision knife 13, U-nail backing ring 14, inner comprehensive cover 15, hook 15-1, U-nail groove 15-2, locking ring 16, screw threads 16-1, inner ring 17, positioning platform 17-1, blood vessel docking groove 17-2, U-nail ring pad groove 17-3, upper cover 18, screw threads 18-1, positioning groove 18-2, positioning groove 18-3.

Refer to the built-in or valgus graduated circumcision device.

FIG. 19 is an overall view of the flat-head circumcision device of the present invention;

FIG. 20 is a structural view of the flat-head circumcision device of the present invention;

FIG. 21is a stereoscopic view of the flat-head circumcision device of the present invention;

FIG. 22 is an overall view of the oblique-head circumcision device of the present invention;

FIG. 23 is a structural view of the oblique-head circumcision device of the present invention;

FIG. 24 is a stereoscopic view of the oblique-head circumcision device of the present invention;

FIG. 25 is a type of a graduation label annularly arranged on a comprehensive cover;

FIG. 26 is a schematic diagram of the position of a nail cabin of a flat-head built-in circumcision device;

FIG. 27 is a schematic diagram of the position of a nail cabin of an oblique-head built-in circumcision device;

FIG. 28 is an overall view of the circumcision device of the present invention;

FIG. 29 is a structural view of the circumcision device of the present invention;

FIG. 30 is an exploded view of a valgus circumcision stapler; and

FIG. 31 is a type of a graduation label annularly arranged on a comprehensive cover.

Refer to the prepuce circumcision stapler with an indication device, which is, more particularly, of a built-in type and a valgus type.

FIG. 32 is an overall view of a flat-head prepuce circumcision stapler of the present invention;

FIG. 33 is a structural view of the flat-head prepuce circumcision stapler of the present invention;

FIG. 34 is a stereoscopic view of the flat-head prepuce circumcision stapler of the present invention;

FIG. 35 is an overall view of an oblique-head prepuce circumcision stapler of the present invention;

FIG. 36 is a structural view of the oblique-head prepuce circumcision stapler of the present invention;

FIG. 37 is a stereoscopic view of the oblique-head prepuce circumcision stapler of the present invention;

FIG. 38 is a schematic diagram of a position of a nail cabin of the flat-head built-in prepuce circumcision stapler;

FIG. 39 is a schematic diagram of a position of a nail cabin of the oblique-head built-in prepuce circumcision stapler;

FIG. 40 is a schematic diagram of a convex structure pre-disposed on the comprehensive cover;

FIG. 41 is a schematic diagram of a groove structure pre-disposed on the comprehensive cover;

FIG. 42 is a schematic diagram of a convex point structure pre-disposed on the comprehensive cover;

FIG. 43 is a schematic diagram of a line structure pre-disposed on the comprehensive cover;

FIG. 44 is a schematic structural diagram of a prefabricated protrusion ferrule;

FIG. 45 is a schematic structural diagram of a prefabricated groove ferrule;

FIG. 46 is a schematic structural diagram of a prefabricated convex point ferrule;

FIG. 47 is a schematic structural diagram of a prefabricated line ferrule;

FIG. 48 is a schematic structural diagram of a prefabricated protrusion sticky label;

FIG. 49 is a schematic structural diagram of a prefabricated groove sticky label;

FIG. 50 is a schematic structural diagram of a prefabricated convex point sticky label;

FIG. 51 is a schematic structural diagram of a prefabricated line sticky label;

FIG. 52 is an enlarged schematic diagram of a part B in FIG. 33;

FIG. 53 is a partially enlarged diagram of a U-nail top ring in FIG. 38;

FIG. 54 is a partially enlarged diagram of a prepuce circumcision cover in FIG. 38;

FIG. 55 is a schematic structural diagram of a prefabricated protrusion ferrule;

FIG. 56 is a schematic structural diagram of a prefabricated groove ferrule;

FIG. 57 is a schematic structural diagram of a prefabricated convex point ferrule;

FIG. 58 is a schematic structural diagram of a prefabricated line ferrule;

FIG. 59 is a schematic structural diagram of a prefabricated protrusion sticky label;

FIG. 60 is a schematic structural diagram of a prefabricated groove sticky label;

FIG. 61 is a schematic structural diagram of a prefabricated convex point sticky label;

FIG. 62 is a schematic structural diagram of a prefabricated line sticky label;

FIG. 63 is an overall view of the prepuce circumcision stapler of the present invention;

FIG. 64 is a structural view of the prepuce circumcision stapler of the present invention;

FIG. 65 is an exploded view of the valgus prepuce circumcision stapler;

FIG. 66 is an enlarged view of the upper half part of the valgus prepuce circumcision stapler in FIG. 65;

FIG. 67 is an enlarged view of the lower half part of the valgus prepuce circumcision stapler in FIG. 65;

FIG. 68 is an enlarged view of an inner ring;

FIG. 69 is an enlarged view of an upper cover;

FIG. 70 is an enlarged view of a part E in FIG. 64;

FIG. 71 is an exploded view of the valgus prepuce circumcision stapler;

FIG. 72 is a structural view of the valgus prepuce circumcision stapler;

FIG. 73 is an enlarged view of a part B in FIG. 72;

FIG. 74 is a main view of the valgus prepuce circumcision stapler;

FIG. 75 is an enlarged view of a U-nail washer; and

FIG. 76 is an enlarged view of an opening knob.

Refer to the operation-visible circumcision stapler.

FIG. 77 is an overall view of a flat-head circumcision stapler of the present invention;

FIG. 78 is a structural view of the flat-head circumcision stapler of the present invention;

FIG. 79 is an enlarged diagram of portion B in FIG. 78;

FIG. 80 is a stereoscopic view of the flat-head circumcision stapler of the present invention;

FIG. 81 is an overall view of an oblique-head circumcision stapler of the present invention;

FIG. 82 is a structural view of the oblique-head circumcision stapler of the present invention;

FIG. 83 is a stereoscopic view of the oblique-head circumcision stapler of the present invention;

FIG. 84 is a diagram of the position of a nail cabin of a flat-head built-in circumcision stapler;

FIG. 85 is a partial enlarged view of a U-nail top ring in FIG. 84;

FIG. 86 is a partial enlarged view of a circumcision cover in FIG. 84;

FIG. 87 is a diagram of the position of a nail cabin of an oblique-head built-in circumcision stapler.

### SPECIFIC DESCRIPTION OF THE EMBODIMENTS

Refer to the blood vessel docking apparatus of a built-in or valgus circumcision device.

A preferable embodiment of multiple implementations of the present invention is described in detail in the following according to the figures.

An embodiment of the built-in type is described in the following.

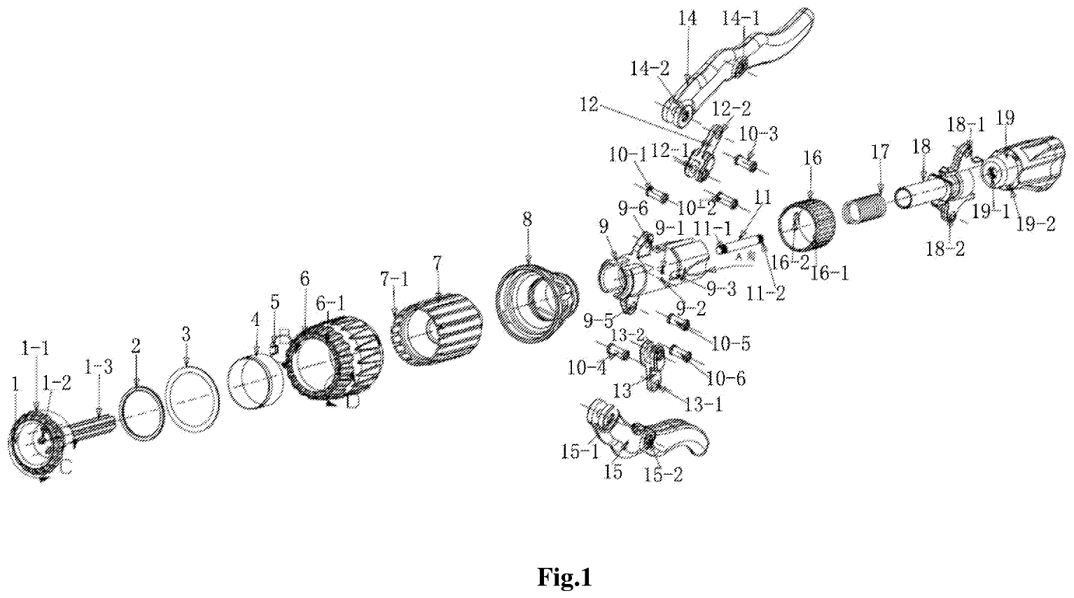

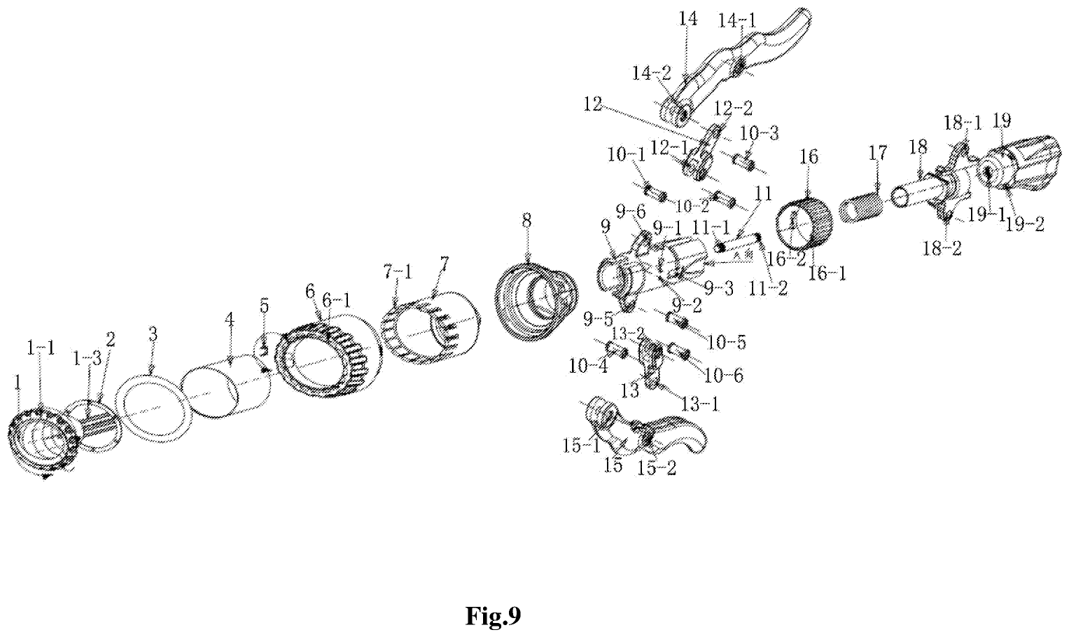

FIGS. 1 and 9 are exploded views of the built-in circumcision device in a flat-head type and an oblique-head type respectively. These drawings respectively show a circumcision cover 1, a blood vessel corresponding groove 1-1, a negative pressure hole 1-2, a positioning rod 1-3, a positioning groove 1-3-1, a U-nail seat 1-4, a circumcision knife washer 2, a U-nail backing ring 3, a circumcision knife 4, a U-nail 5, a comprehensive cover 6, a U-nail groove 6-1, a blood vessel docking groove 6-2, a U-nail top ring 7, a U-nail top column 7-1, a positioning hole 7-2, a positioning groove 7-2-1, a lower comprehensive cover 8, a connecting block 9, a closing mark 9-1, an opening mark 9-2, an opening guide groove 9-3, a positioning convex point 9-4, a pin hole 9-5, a pin hole 9-6, a pin 10-1, a pin 10-2, a third pin 10-3, a fourth pin 10-4, a fifth pin 10-5, a sixth pin 10-6, a connecting rod 11, screw threads 11-1, screw threads 11-2, a small connecting rod 12, a pin hole 12-1, a pin hole 12-2, a small connecting rod 13, a pin hole 13-1, a pin hole 13-2, a handle 14, a pin hole 14-1, a pin hole 14-2, a handle 15, a pin hole 15-1, a pin hole 15-2, an opening knob 16, a pointer 16-1, a sliding block 16-2, a spring 17, a sliding rod 18, a pin hole 18-1, a pin hole 18-2, a knob 19, a connecting screw threads 19-1, a positioning concave point 19-2. The related driving components, actuating components and circumcision components and so on are common structures in the art. Refer to the drawings, the position relations and connecting relations are both clear, and the various combinations in the prior art can be adopted, so the details will not be repeated here. The blood vessel docking apparatus of the circumcision device comprises a circumcision cover 1, a comprehensive cover 6, a blood vessel corresponding groove 1-1, a blood vessel docking groove 6-2, a U-nail top ring 7 and a lower comprehensive cover 8; wherein the circumcision cover 1 and the comprehensive cover 6 are used for assembling to each other; a plurality of blood vessel corresponding grooves 1-1 is disposed along the periphery of the front end of the circumcision cover 1, a plurality of blood vessel docking grooves 6-2 is disposed along the periphery of the front end of the comprehensive cover 6; the back end of the circumcision cover 1 is provided with a positioning rod 1-3 in matching connection with the positioning groove 1-3-1 of the U-nail top ring 7; and the back end of the comprehensive cover 6 is connected to the lower comprehensive cover 8.

A front end of the blood vessel corresponding groove 1-1 is close to an annular top end of a circumcision cover 1, and a back end of the blood vessel corresponding groove 1-1 is away from the annular top end of the circumcision cover 1 backwards relative to the front end of the blood vessel corresponding groove 1-1. The blood vessel corresponding groove 1-1 obliquely extends backwards from the front end of the circumcision cover 1. A top end of the comprehensive cover 6 is provided with an annular outer wall. The blood vessel docking groove 6-2 is disposed on an outer surface of the annular outer wall. A front end of the blood vessel docking groove 6-2 is slightly higher than the top end of the annular outer wall. A back end of the blood vessel docking groove 6-2 is away from the top end of the annular outer wall backwards relative to the front end of the blood vessel docking groove 6-2. The blood vessel docking groove 6-2 obliquely extends backwards from the top end of the annular outer wall.

The plurality of blood vessel corresponding grooves 1-1 corresponds to a blood vessel docking groove 6-2 and a U-nail groove 6-1, and/or a U-nail 5 in a U-nail groove 6-1 one by one. An arc-shaped transition surface is arranged between the blood vessel corresponding grooves 1-1 and the annular top end of the circumcision cover 1. An arc-shaped transition surface is arranged between the blood vessel corresponding grooves 1-1 and convex plates formed among the blood vessel corresponding grooves 1-1.

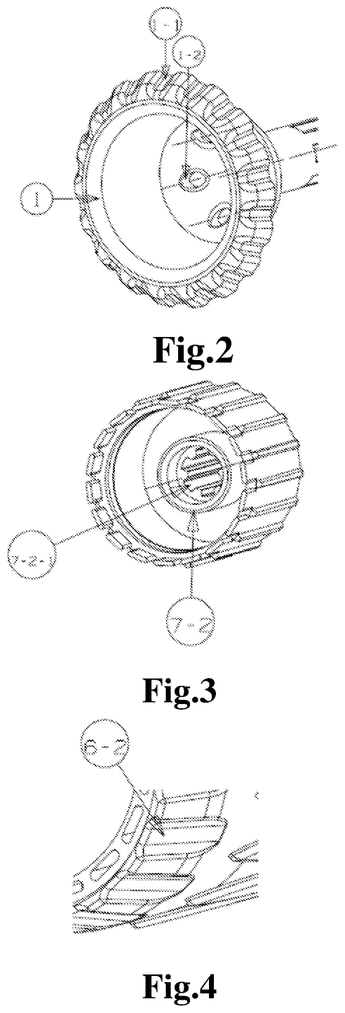

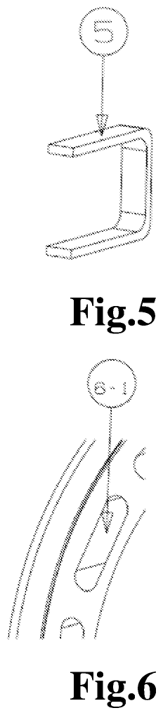

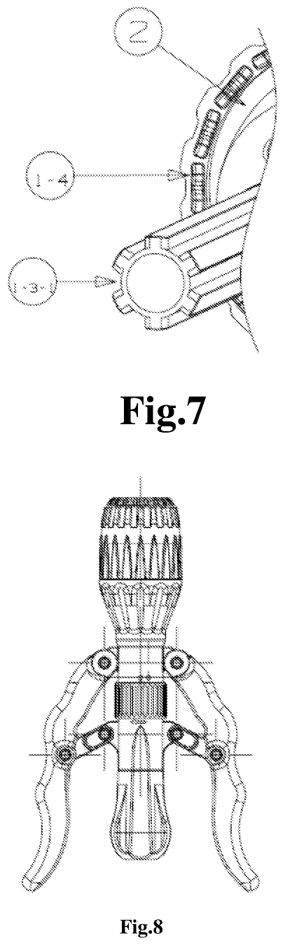

FIG. 2-8 are partial enlarged views of a circumcision cover, a U-nail top ring, a comprehensive cover, a U-nail, a U-nail groove and a lower end of the circumcision cover of a flat-head type circumcision device, an overall view of the built-in flat-head circumcision device arranged with a blood vessel docking apparatus in the built-in type, wherein the circumcision cover is provided with a negative pressure hole 1-2 for placing a glans penis, a positioning hole 7-2 is disposed in the U-nail top ring 7, the positioning groove 7-2-1 corresponding to a positioning rod 1-3 is disposed in the positioning hole 7-2. FIG. 4 is a preferable setting of a blood vessel docking groove 6-2. The front end of the blood vessel docking groove 6-2 is narrower than the back end. FIG. 6 is a preferable setting of a U-nail groove 6-1. The comprehensive cover 6 is provided with a plurality of U-nail grooves 6-1 for receiving U-nails 5, nail outlets of the U-nail grooves 6-1 are disposed on the top end of the comprehensive cover 6, and the nail outlets of the U-nail grooves 6-1 are disposed inside the annular outer wall. A positioning groove 1-3-1 is disposed on a positioning rod 1-3 of the lower end of the circumcision cover, and cooperates with the positioning hole 7-2 disposed in the U-nail top ring 7. U-nail seats 1-4 corresponding to the U-nails 5 are further disposed along the circumcision cover.

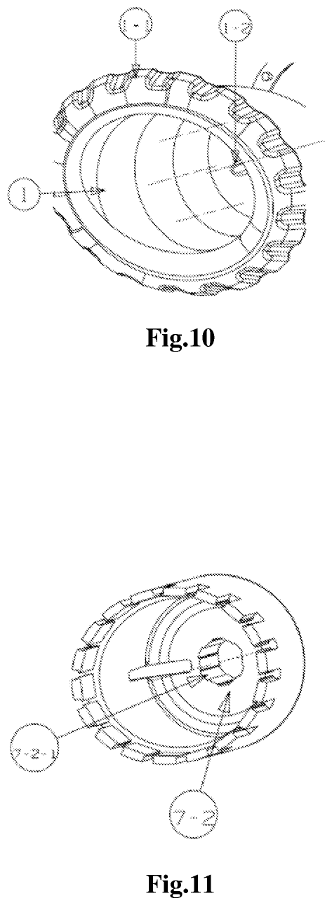

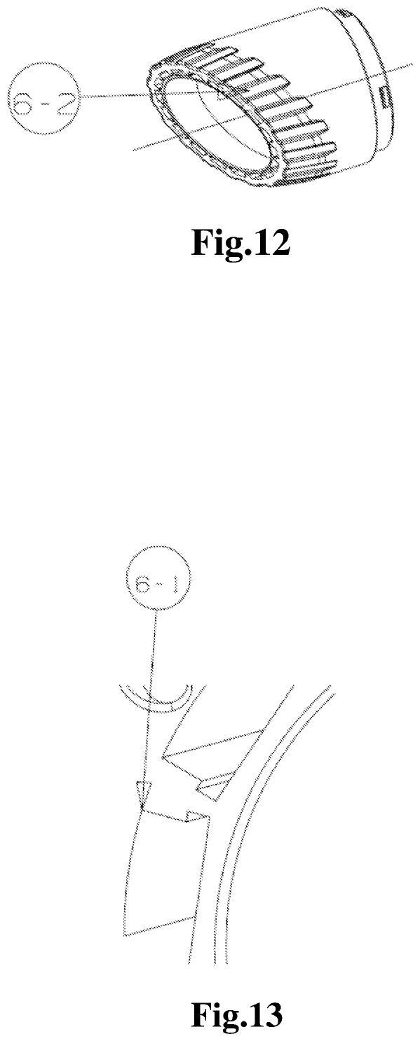

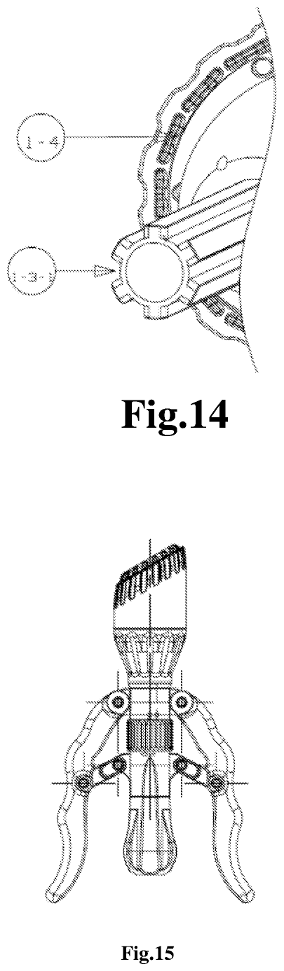

FIGS. 10-15 are partial enlarged views of a circumcision cover, a U-nail top ring, a comprehensive cover, a U-nail groove and a lower end of the circumcision cover of an oblique-head type circumcision device, an overall view of the built-in oblique-head circumcision device arranged with a blood vessel docking apparatus in the built-in type, wherein the circumcision cover is provided with a negative pressure hole 1-2 for placing a glans penis, a positioning hole 7-2 is disposed in a U-nail top ring 7, a positioning groove 7-2-1 corresponding to a positioning rod 1-3 is disposed in a positioning hole 7-2. FIG. 12 is a preferable setting of a blood vessel docking groove 6-2, wherein the widths of the front and the back of the blood vessel docking groove 6-2 are the same. FIG. 13 is a preferable setting of a U-nail groove 6-1. The comprehensive cover 6 is provided with a plurality of U-nail grooves 6-1 for receiving U-nails 5. Nail outlets of the U-nail grooves 6-1 are disposed on the top end of the comprehensive cover 6. The nail outlets of the U-nail grooves 6-1 are disposed inside the annular outer wall. A positioning groove 1-3-1 is disposed on a positioning rod 1-3 of the lower end of the circumcision cover, and cooperates with the positioning hole 7-2 disposed in the U-nail top ring 7. U-nail seats 1-4 corresponding to the U-nails 5 are further disposed along the circumcision cover.

An embodiment of the valgus type is described in the following.

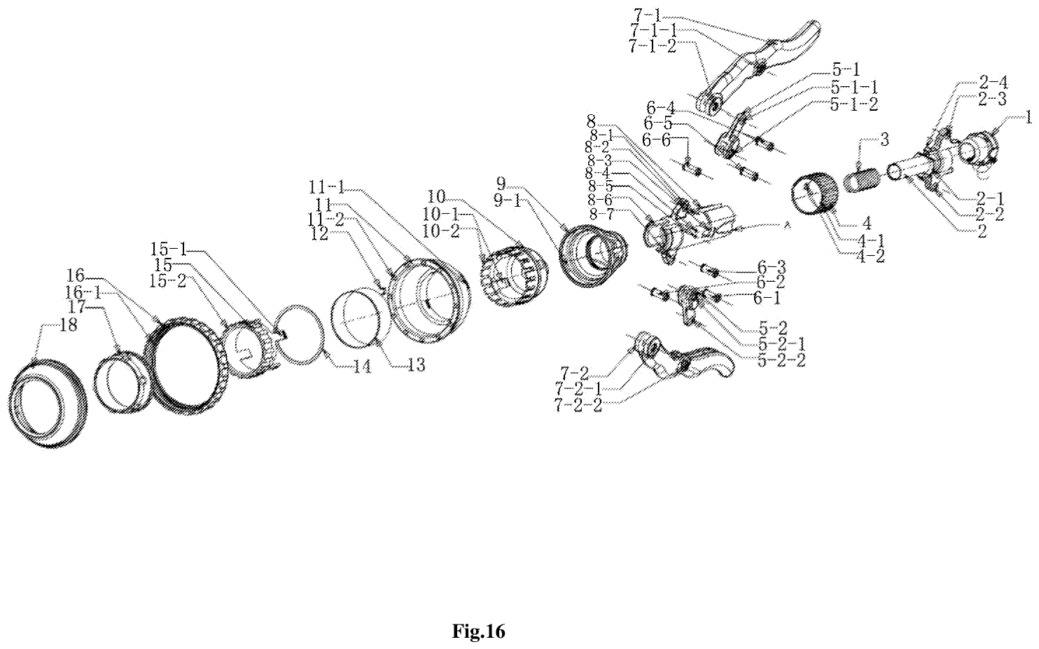

FIG. 16 is an exploded view of the valgus circumcision device arranged with a blood vessel positioning device. The drawing shows a plug 1, a positioning concave point 1-1, a sliding rod 2, a spring supporting platform 2-1, a pin hole 2-2, a pin hole 2-3, a hook 2-4, a spring 3, an opening knob 4, a pointer 4-1, a sliding block 4-2, a connecting rod 5, a small connecting rod 5-1, a pin hole 5-1-1, a pin hole 5-1-2, a small connecting rod 5-2, a pin hole 5-2-1, a pin hole 5-2-2, a pin 6-1, a pin 6-2, a pin 6-3, a pin 6-4, a pin 6-5, a pin 6-6, a handle 7-1, a pin hole 7-1-1, a pin hole 7-1-2, a handle 7-2, a pin hole 7-2-1, a pin hole 7-2-2, a connecting block 8, a clamping platform 8-1, a pin hole 8-2, an opening guide groove 8-3, a closing mark 8-4, an opening mark 8-5, a hook 8-6, a pin hole 8-7, a positioning convex point 8-8, a comprehensive cover 9, a clamping groove 9-1, a U-nail top ring 10, a groove 10-1, a U-nail top column 10-2, a lower cover 11, a clamping platform 11-1, a positioning platform 11-2, a U-nail 12, a circumcision knife 13, a U-nail backing ring 14, an inner comprehensive cover 15, a hook 15-1, a U-nail groove 15-2, a locking ring 16, screw threads 16-1, an inner ring 17, a positioning platform 17-1, a blood vessel docking groove 17-2, a U-nail ring pad groove 17-3, an upper cover 18, screw threads 18-1, a positioning groove 18-2, and a positioning groove 18-3. The related driving components, actuating components and circumcision components and so on are common structures in the art. Refer to the drawings, the position relations and connecting relations are both clear, and the various combinations in the prior art can be adopted, so the details will not be repeated here. The blood vessel docking apparatus of the valgus circumcision device comprises a comprehensive cover 9, a U-nail top ring 10, a lower cover 11, an inner comprehensive cover 15, an inner ring 17, an upper cover 18 and a blood vessel docking groove 17-2. The inner ring 17 cooperates with the inner comprehensive cover 15. A plurality of blood vessel docking grooves 17-2 is disposed along the periphery of the back end of the inner ring 17. The U-nail top ring 10 is assembled and matched with the inner comprehensive cover 15 so as to push a U-nail 12 out of a U-nail groove 15-2. The upper cover 18 and the lower cover 11 are used for assembling and connecting with each other. The inner ring 17 is disposed in the upper cover 18. The inner comprehensive cover 15 is partially disposed in the lower cover 11. A front end of the comprehensive cover 9 is connected to a back end of the lower cover 11. The U-nail top ring 10 is partially disposed in the comprehensive cover 9.

The inner comprehensive cover 15 is provided with a plurality of U-nail grooves 15-2 for receiving U-nails 12, and the U-nail grooves 15-2 are disposed along the periphery of the front end of the inner comprehensive cover 15. The nail outlets of the U-nail grooves 15-2 are disposed close to the top end of the inner comprehensive cover 15. The positions of a plurality of blood vessel docking grooves 17-2 are corresponding to those of the U-nail grooves 15-2 one by one. The blood vessel docking grooves 17-2 are pre-disposed on the inner ring 17. The back ends of the blood vessel docking grooves 17-2 are flush with the back end of the inner ring 17. The front ends of the blood vessel docking grooves 17-2 are away from the annular back end of the inner ring 17 forwards relative to the back ends of the blood vessel docking grooves 17-2.

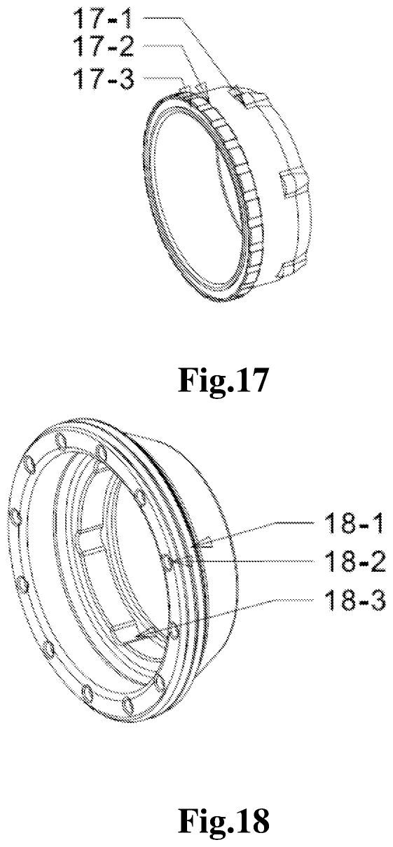

FIGS. 17-18 are partial enlarged views of an inner ring and an upper cover. The front end of the inner ring 17 is disposed with positioning platforms 17-1 for positioning, the periphery of the back end thereof is provided with blood vessel docking grooves 17-2, and the back end face thereof is disposed with a U-nail ring pad groove 17-3 for corresponding to U-nails. The outer periphery of the back end of the upper cover 18 is disposed with screw threads 18-1 and positioning grooves 18-2 for connecting with a lower cover 11 and a locking ring 16. The central part close to the front end of the upper cover is provided with positioning grooves 18-3 for corresponding to the positioning platforms 17-1.

Exemplary descriptions are given for the present invention with reference to the drawings in the above. Obviously, the present invention is not limited to the above implementation manners. Any improvement using the methods, concepts and technical solutions of the present invention or direct application of the present invention to other occasions without modifications shall fall into the protection scope of the present invention.

Refer to the built-in or valgus graduated circumcision device.

A preferred embodiment of multiple implementations of the present invention is described in detail below according to the drawings.

The following is an embodiment of the built-in type.

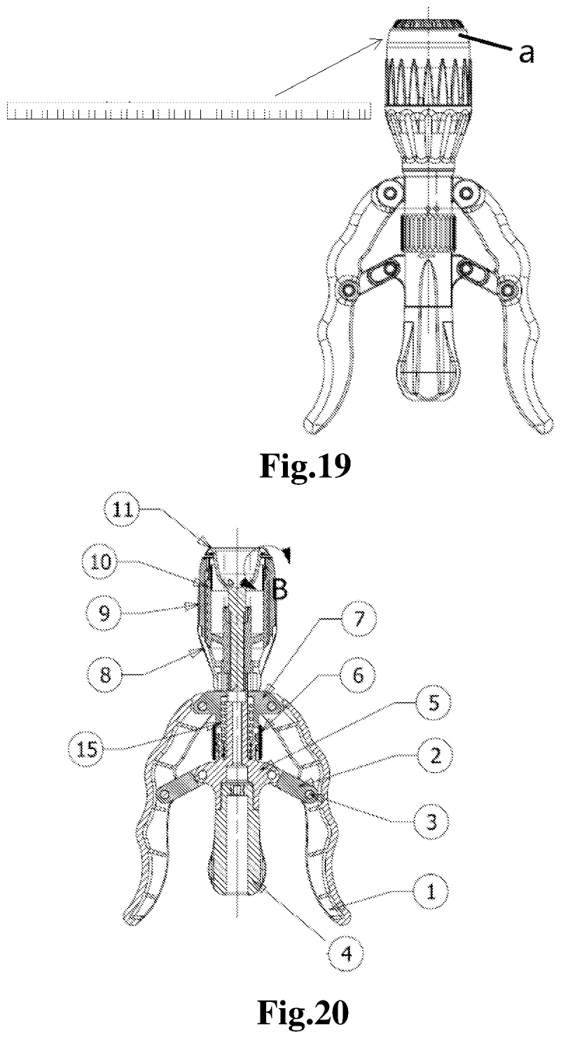

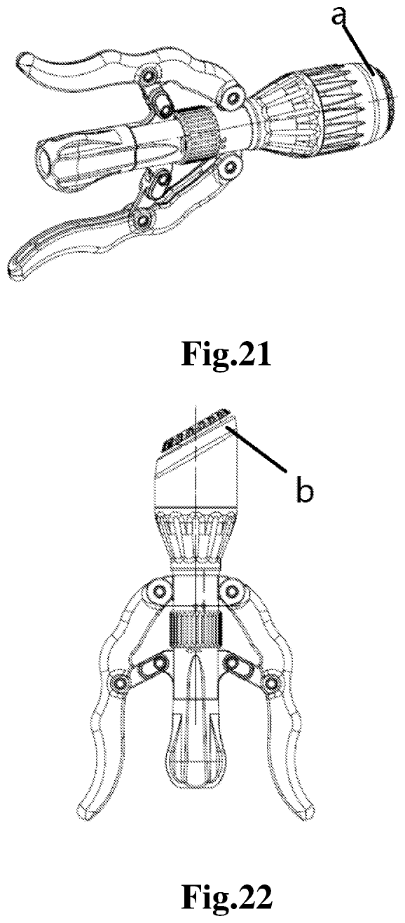

FIG. 19 is an overall view of the flat-head circumcision device of the present invention. A position indicated by a in the figure is a preferred position for arrangement of a graduation device. After release paper on the back is peeled off, a graduation label is annularly adhered to a periphery of the end, close to a circumcision cover, of a comprehensive cover by means of an adhesive layer on the back. FIG. 20 is a structural view of the flat-head circumcision device of the present invention. The flat-head circumcision device comprises handles 1, connection rods 2, rivets 3, a knob 4, a sheathed core 5, an opening knob 6, sheathed handles 7, a lower cover 8, a comprehensive cover 9, a thimble 10, a circumcision cover 11, a circumcision knife 12, a first washer 13, a third washer 14, springs 15 and a second washer 16. Relevant driving components, actuating components and circumcision components are common structures in the prior art. With reference to the drawing, position relationships and connection relationships thereof are both clear, and the details will not be repeated here. FIG. 21 is a stereoscopic view of the flat-head circumcision device of the present invention. A position indicated by a in the figure is a preferred position for arrangement of a graduation device,

This built-in graduated circumcision device comprises a comprehensive cover 9, a circumcision cover 1 and a graduation device, wherein the comprehensive cover 9 and circumcision cover 1 are assembled to and cooperate with each other; and the graduation device is arranged on the comprehensive cover 9. The graduation device comprises a ring of graduations annularly arranged on a periphery of the end, close to the circumcision cover 1, of the comprehensive cover 9.

A plurality of U-nail grooves which constitutes a ring is arranged in the comprehensive cover 9. Graduation marks on the graduation device are arranged to be in one-to-one correspondence with the U-nail grooves and/or interval between the U-nail grooves.

A ring of blood vessel docking grooves are annularly arranged on an outer surface of the comprehensive cover 9, and the graduation marks on the graduation device are arranged to be in one-to-one correspondence with the blood vessel docking grooves and/or intervals between the blood vessel docking grooves. A ring of blood vessel corresponding grooves 1-1 are annularly arranged on an outer surface of the circumcision cover 1, and the graduation marks on the graduation device are arranged to be in one-to-one correspondence with the blood vessel corresponding 1-1 grooves and/or the interval between the blood vessel corresponding grooves 1-1.

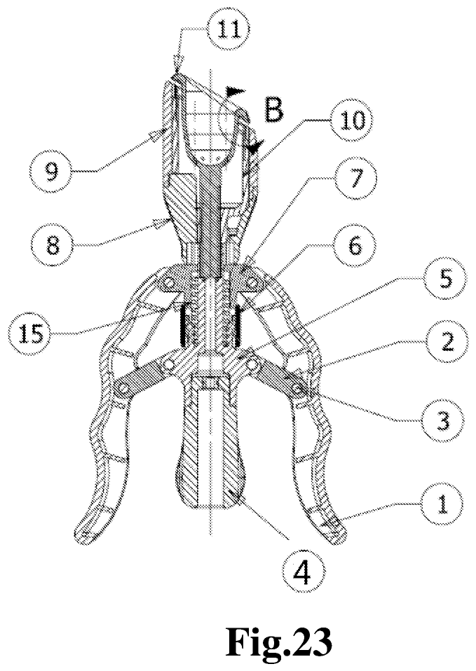

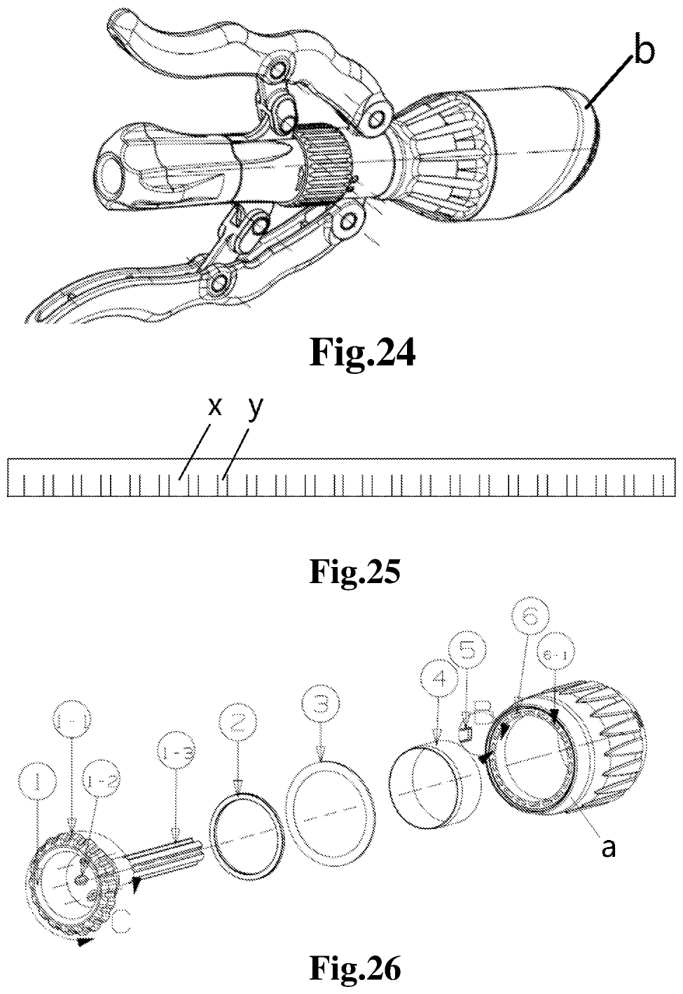

FIG. 22 is an overall view of the oblique-head circumcision device of the present invention. A position indicated by b in the figure is a preferred position for arrangement of a graduation device. FIG. 23 is a structural view of the oblique-head circumcision device of the present invention. The oblique-head circumcision device comprises handles 1, connection rods 2, rivets 3, a knob 4, a sheathed core 5, an opening knob 6, sheathed handles 7, a lower cover 8, a comprehensive cover 9, a thimble 10, a circumcision cover 11, a circumcision knife 12, a first washer 13, a third washer 14, springs 15 and a second washer 16. Relevant driving components, actuating components and circumcision components are common structures in the prior art. With reference to the drawing, position relationships and connection relationships thereof are both clear, and the details will not be repeated here. FIG. 24 is a stereoscopic view of the oblique-head circumcision device of the present invention. A position indicated by b in the figure is a preferred position for arrangement of a graduation device.

FIG. 25 is a type of a graduation label annularly arranged on a comprehensive cover. Graduations are printed on the front thereof, and an adhesive surface and release paper are arranged on the back thereof. The adhesive surface is used for adhesion at the position a or the position b described above after the release paper is peeled off. Intervals x among graduation marks of the graduations on the front thereof correspond to U-nail grooves, U-nails in the U-nail grooves, blood vessel docking grooves, or blood vessel corresponding grooves. Intervals y among the graduation marks of the graduation on the front thereof correspond to intervals among the U-nail grooves, intervals among the blood vessel docking grooves, or intervals among the blood vessel corresponding grooves.

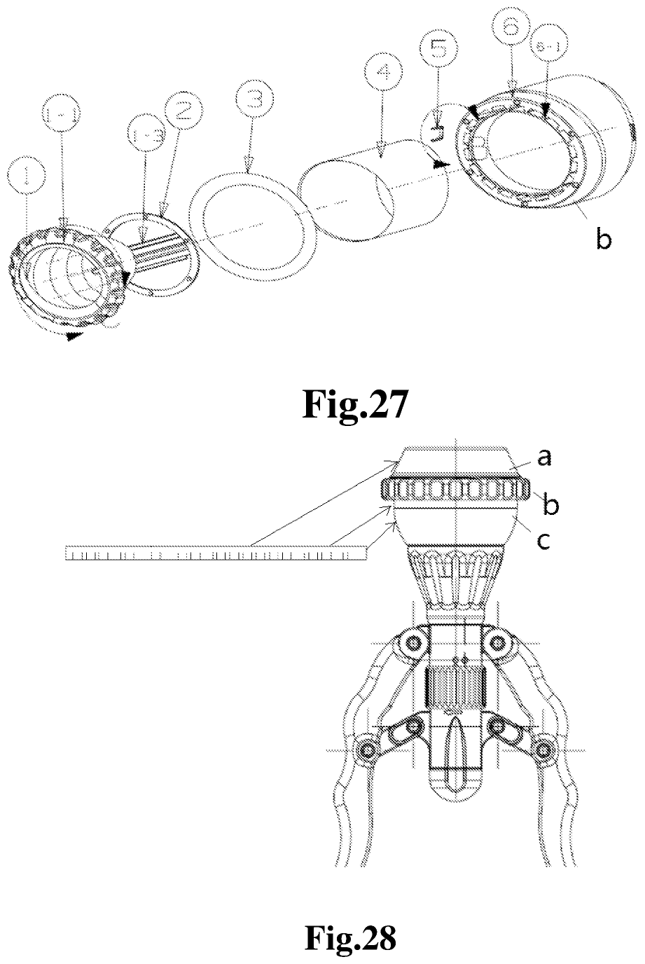

FIGS. 26 and 27 are schematic views of positions of nail cabins of the flat-head and oblique-head built-in circumcision devices, respectively. In the figures, 1 represents a circumcision cover, 1-1 blood vessel corresponding grooves, 1-2 a negative pressure, 1-3 a positioning rod, 1-3-1 a positioning groove, 1-4 U-nail seats, 2 a circumcision knife washer, 3 U-nail backing ring, 4 circumcision knife, 5 U-nails, 6 a comprehensive cover, and 6-1 U-nail grooves. Relevant driving components, actuating components and circumcision components are common structures in the prior art. With reference to the drawings, position relationships and connection relationships thereof are both clear, and the details will not be repeated here.

The following is an embodiment of the valgus type.

FIG. 28 is an overall view of the valgus circumcision device of the present invention. A position indicated by a, b, or c in the figure is a preferred position for arrangement of a graduation device. After release paper on the back is peeled off, a graduation label is annularly adhered to a periphery of the end of an upper cover and/a lower cover, or a periphery of a locking ring.

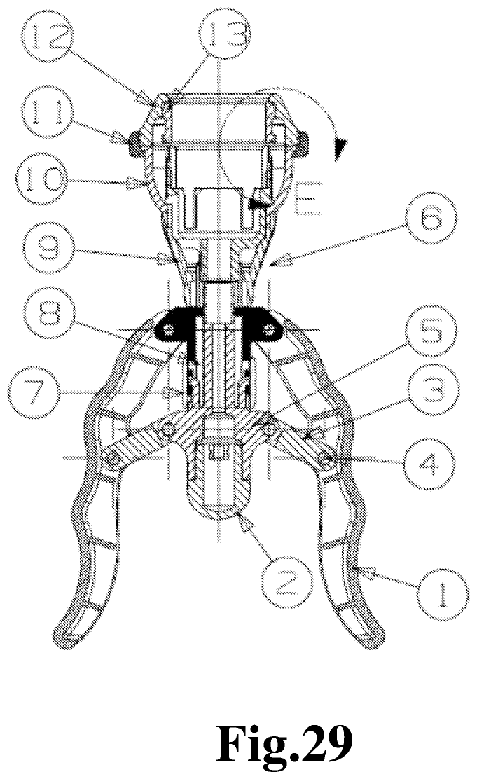

FIG. 29 is a structural view of the valgus circumcision device of the embodiment. In the figure, 1 represents handles, 2 a plug, 3 small connection rods, 4 pins, 5 a sliding rod, 6 a connection block, 7 an opening knob, 8 springs, 9 a comprehensive cover, 10 a lower cover, 11 a locking ring, 12 an upper cover, 13 an inner ring, 14 an inner comprehensive cover, 15 an annular blade, and 16 a first washer/U-nail backing ring. Relevant driving components, actuating components and circumcision components are common structures in the prior art. With reference to the drawing, position relationships and connection relationships thereof are both clear, and the details will not be repeated here.

A valgus graduated circumcision device comprises an upper cover 12, a lower cover 10, a locking ring 11, and a graduation device, wherein the upper cover 12 and the lower cover 10 are assembled to and cooperate with each other; the locking ring 11 is used for locking the upper cover 12 and the lower cover 10; the graduation device is arranged on the upper cover 12, the lower cover 10 or the locking ring 11. The graduation device comprises a ring of graduations annularly arranged on a periphery of the end, close to the lower cover 10, of the upper cover 12, or arranged on a periphery of the end, close to the upper cover 12, of the lower cover 10. Graduation marks on the graduation device are arranged to be in one-to-one correspondence with U-nail grooves and/or intervals among the U-nail grooves. The valgus graduated circumcision device further comprises an inner comprehensive cover 14 arranged in the lower cover 10, wherein a plurality of U-nail grooves are annularly arranged on a periphery of the inner comprehensive cover 14 and constitute a ring.

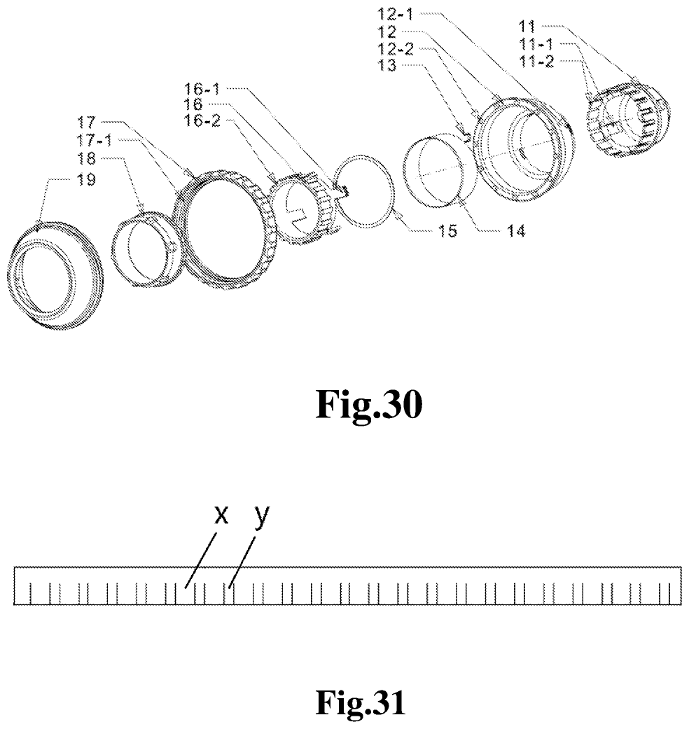

FIG. 30 is an exploded view of a valgus circumcision stapler. In the figure, 11 represents a U-nail top ring, 11-1 grooves, 11-2 U-nail top columns, 12 an upper cover, 12-1 a clamping platform, 12-2 a positioning platform, 13 U-nails, 14 a circumcision knife, 15 a U-nail backing ring/a first washer, 16 an inner comprehensive cover, 16-1 a hook, 16-2 U-nail grooves, 17 a locking ring, 17-1 screw threads, 18 an inner ring, 18-1 a positioning platform, 18-2 blood vessel docking grooves, 18-3 U-nail ring backing grooves, and 19 an upper cover. Relevant driving components, actuating components and circumcision components are common structures in the prior art. With reference to the drawing, position relationships and connection relationships thereof are both clear, and the details will not be repeated here. A position indicated by a in the figure is a preferred position of the upper cover for arrangement of the graduation device. A position indicated by b in the figure is a preferred position of the locking ring for arrangement of the graduation device. A position indicated by c in the figure is a preferred position of the lower cover for arrangement of the graduation device.

FIG. 31 is a type of a graduation label annularly arranged on an upper cover, a lower cover or a locking ring. Graduations are printed on the front thereof, an adhesive surface and release paper are arranged on the back thereof. The adhesive surface is used for adhesion at the position a or b or c described above after the release paper is peeled off. Intervals x among graduation marks of the graduations on the front thereof correspond to U-nail grooves and U-nails in the U-nail grooves. Intervals y among the graduation marks of the graduation on the front thereof correspond to intervals among the U-nail grooves.

Exemplary descriptions are given for the present invention with reference to the drawings in the above. Obviously, the present invention is not limited to the above implementation manners. Any improvement using the methods, concepts and technical solutions of the present invention or direct application of the present invention to other occasions without modifications shall fall into the protection scope of the present invention.

Refer to the prepuce circumcision stapler with an indication device, which is, more particularly, of a built-in type and a valgus type.

Preferred embodiments of multiple implementations of the present invention are described in detail below according to the drawings.

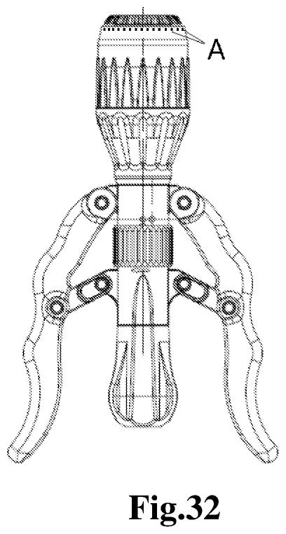

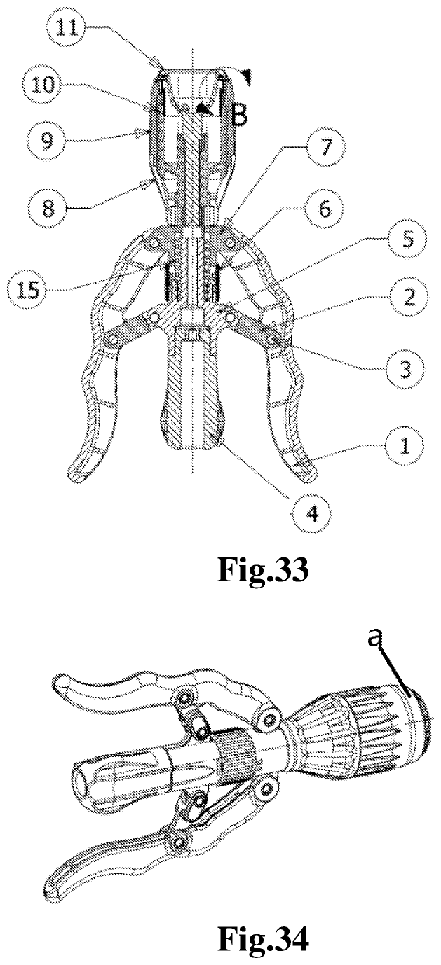

Regarding the built-in type, FIG. 32 is an overall view of a flat-head prepuce circumcision stapler of the present invention. In FIG. 32, an indication point A is a convex point which is arranged on the periphery of one end, close to a prepuce circumcision cover, of a comprehensive cover and can be contacted and perceived by an operator, such that the position of the indication point can be determined through vision or tactile sense during surgery, thereby accurately corresponding to a position of a U-nail groove or a nail cabin. FIG. 33 is a structural view of a flat-head prepuce circumcision stapler of the present invention. FIG. 52 is a partially enlarged view. The flat-head prepuce circumcision stapler comprises handles 1, connecting rod 2, rivets 3, a knob 4, a sheathed core 5, an opening knob 6, a sheathed handle 7, a lower sleeve 8, a comprehensive cover 9, a thimble 10, a prepuce circumcision cover 11, a circumcision knife 12, a first washer 13, a third washer 14, a spring 15 and a second washer 16. The prepuce circumcision cover 11 is inserted from the top end of the comprehensive cover 9. The annular lower end at the upper part of the prepuce circumcision cover 11 serves to clamp the prepuce in cooperation with the comprehensive cover 9. The lower end of the comprehensive cover 9 is connected with the lower sleeve 8. A U-nail top ring is arranged in the comprehensive cover 9 and the lower cover 8. A thimble 10 is arranged on the top of the U-nail top ring. The lower end of the lower sleeve 8 is connected to the sheathed handle 7. The lower end of the sheathed handle 7 is connected to the sheathed core 5. The knob 4 which serves as a plug is arranged at the tail end of the sheathed core 5. The circumcision knife is arranged in the U-nail top ring. The first washer 13, the second washer 16 and the third washer 14 are arranged at the top end of the U-nail groove, an outer ring and an inner ring of a circumcision surface of the prepuce circumcision cover respectively. The handles 1, the connecting rods 2 and the rivets 3 constitute a driving component which is connected to the sheathed core 5 and the sheathed handle 7 and is used for driving the U-nail top ring and/or the circumcision knife 12, thereby finishing the circumcision and suturing. The opening knob 6 sleeves the sheathed core 5 and is used for locking and unlocking the driving component. FIG. 34 is a stereoscopic view of the flat-head prepuce circumcision stapler of the present invention, in which a position indicated by a is a preferred position for arrangement of an indication device.

The built-in prepuce circumcision stapler with an indication device comprises a comprehensive cover 9, a prepuce circumcision cover 1 and the indication device. The comprehensive cover 9 and the prepuce circumcision cover 1 are assembled to and cooperate with each other. The indication device is arranged on the comprehensive cover 9. The indication device is a ring of protrusions or recesses which are annularly arranged on the periphery of one end, close to the prepuce circumcision cover 1, of the comprehensive cover 9. A plurality of U-nail grooves 6-1 which serves to accommodate U-nails and constitutes a ring is arranged in the comprehensive cover 9. The intervals among the protrusions or recesses of the indication device are arranged to be in one-to-one correspondence with the U-nail grooves 6-1. The width between the two protrusions is equal to the width of the U-nail groove 6-1. The indication device is protrusions or recesses prefabricated on the comprehensive cover 9, wherein the protrusions are convex points.

The protrusions of the convex points can be contacted and perceived by an operator when the prepuce covers or does not cover the protrusions. Preferably, the convex points are circular convex points. A ring of blood vessel docking grooves are annularly arranged on the outer surface of the comprehensive cover 9. The protrusions on the indication device are arranged to be in one-to-one correspondence with the blood vessel docking grooves and/or intervals among the blood vessel docking grooves. A ring of blood vessel corresponding grooves are annularly arranged on the outer surface of the prepuce circumcision cover. The protrusions on the indication device are arranged to be in one-to-one correspondence with the blood vessel corresponding grooves and/or intervals among the blood vessel corresponding grooves.

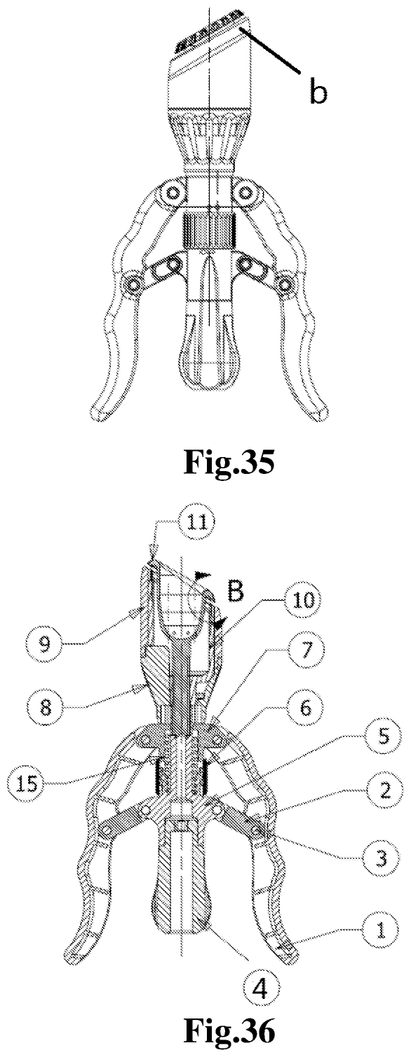

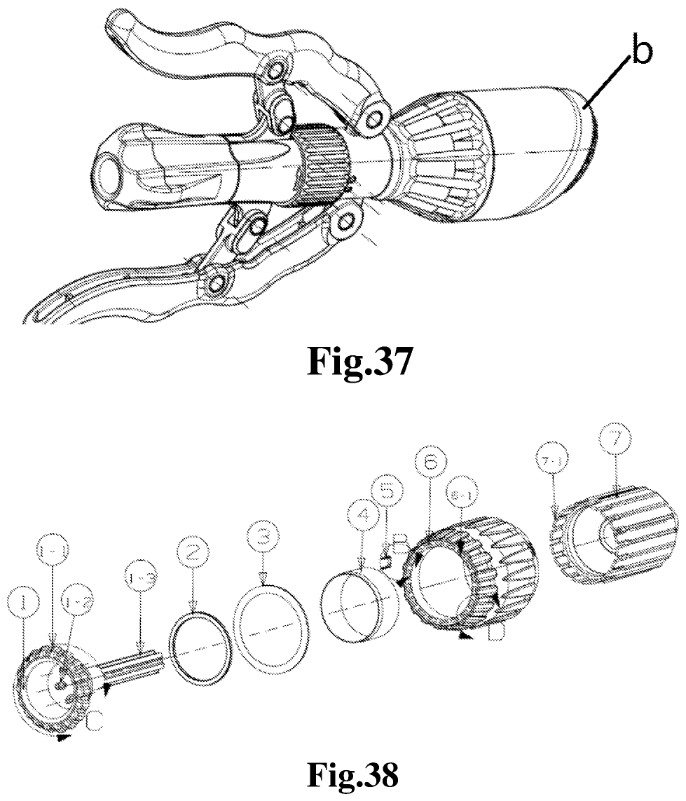

FIG. 35 is an overall view of the oblique-head prepuce circumcision stapler of the present invention, in which a position indicated by b in the figure is a preferred position for arrangement of the indication device. FIG. 36 is a structural view of the oblique-head prepuce circumcision stapler of the present invention. The oblique-head prepuce circumcision stapler comprises handles 1, connecting rods 2, rivets 3, a knob 4, a sheathed core 5, an opening knob 6, a sheathed handle 7, a lower cover 8, a comprehensive cover 9, a thimble 10, a prepuce circumcision cover 11, a circumcision knife 12, a first washer 13, a third washer 14, springs 15 and a second washer 16. With reference to the drawings, position relationships and connection relationships of relevant driving components, the actuating components and circumcision components are both clear, and the details will not be repeated here. FIG. 37 is a stereoscopic view of the oblique-head prepuce circumcision stapler of the present invention, in which a position indicated by b is a preferred position for arrangement of the indication device.

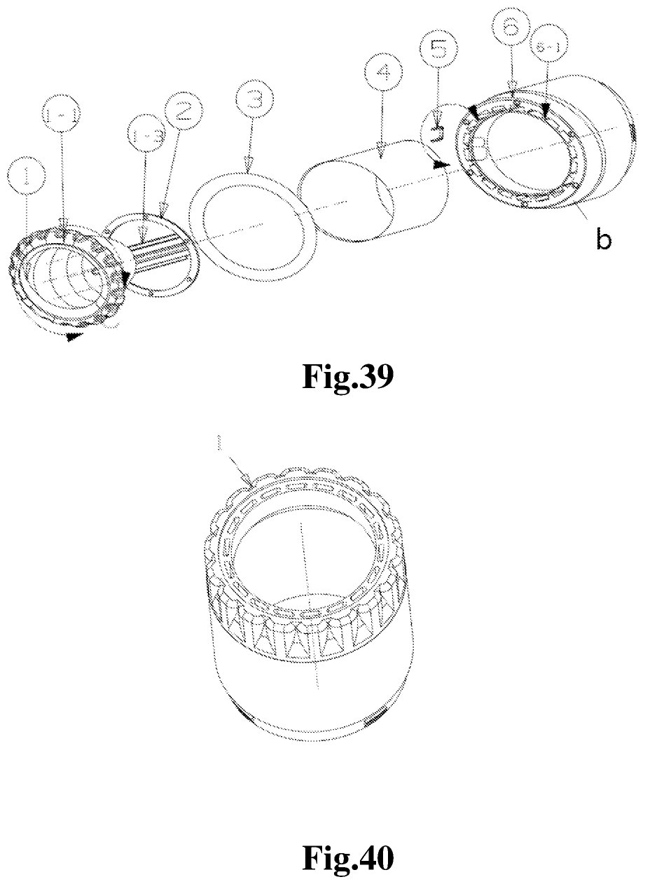

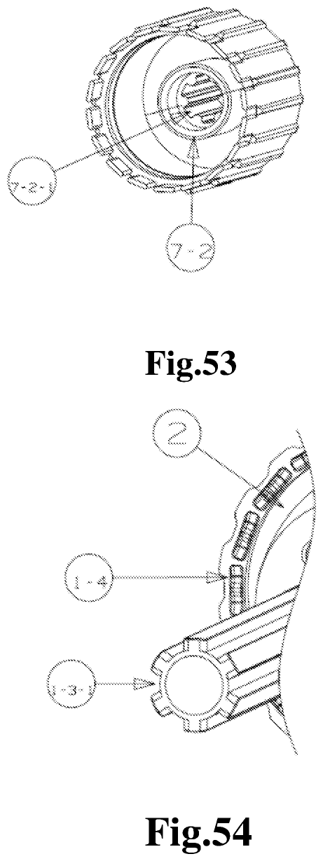

FIG. 38 and FIG. 39 are schematic diagrams of the positions of the nail cabins of the flat-head and oblique-head built-in prepuce circumcision stapler respectively. FIG. 53 and FIG. 54 are partially enlarged views of the U-nail top ring and the prepuce circumcision cover in FIG. 38. In these drawings, reference signs represent the following components: blood vessel corresponding groove 1-1; negative pressure hole 1-2; positioning hole 1-3; positioning groove 1-3-1; U-nail seat 1-4; circumcision knife washer 2; U-nail backing ring 3; circumcision knife 4; U-nail 5; comprehensive cover 6; U-nail groove 6-1; U-nail top ring 7; thimble 7-1; positioning hole 7-2; positioning groove 7-2-1. The prepuce circumcision stapler comprises the comprehensive cover 6, the prepuce circumcision cover 1, the U-nail top ring 7 and the circumcision knife 4. The lower end of the prepuce circumcision cover 1 is inserted into the comprehensive cover 6. The upper end of the prepuce circumcision cover 1 supports the top end of the comprehensive cover 6 and serves to clamp the prepuce in cooperation with the comprehensive cover 6. A plurality of U-nail grooves 6-1 which serves to accommodate U-nails and constitutes a ring is arranged in the comprehensive cover 6. There is an interval between the U-nail grooves 6-1. The U-nail top ring 7 is arranged in the comprehensive cover 6. The top end of the U-nail top ring 7 is provided with thimbles 7-1 which are in one-to-one correspondence to the U-nail grooves 6-1. The circumcision knife 4 is arranged in the U-nail top ring 7. The upper end of the prepuce circumcision cover 1 is provided with blood vessel corresponding grooves 1-1 which are annularly arranged and are in one-to-one correspondence to the U-nail grooves 6-1. The lower end of the prepuce circumcision cover 1 is provided with a positioning rod 1-3. The U-nail top ring 7 is provided with a positioning hole 7-2. The positioning rod 1-3 serves to be inserted and positioned in the positioning hole 7-2, such that the blood vessel corresponding grooves 1-1 on the prepuce circumcision cover 1 are in one-to-one correspondence to the U-nail grooves 6-1. The outer surface of the comprehensive cover 6 is annularly provided with a ring of blood vessel docking grooves or graduations which are in one-to-one correspondence to the U-nail grooves 6-1 and/or the blood vessel corresponding grooves. With reference to the drawings, the position relationships and connection relationships of relevant driving components, the actuating components and circumcision components are both clear, and the details will not be repeated here.

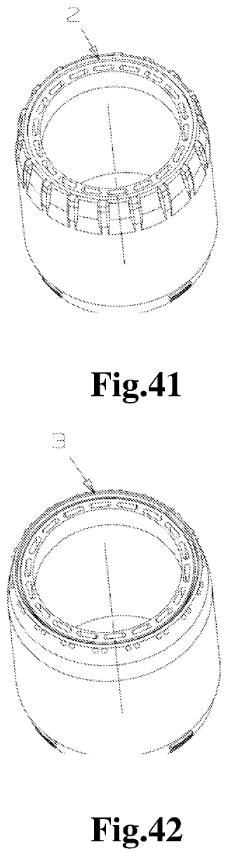

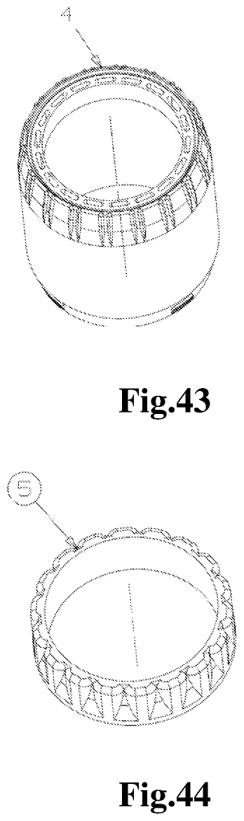

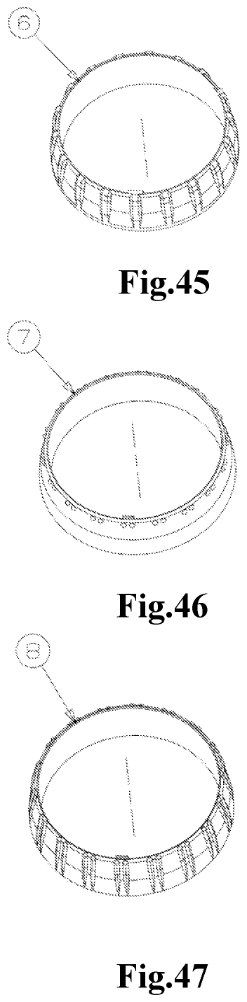

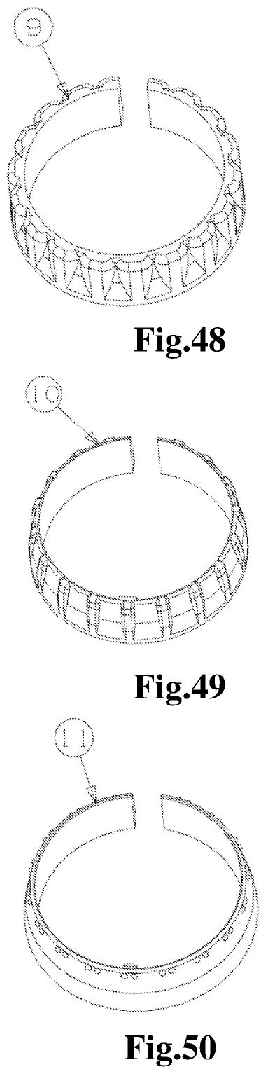

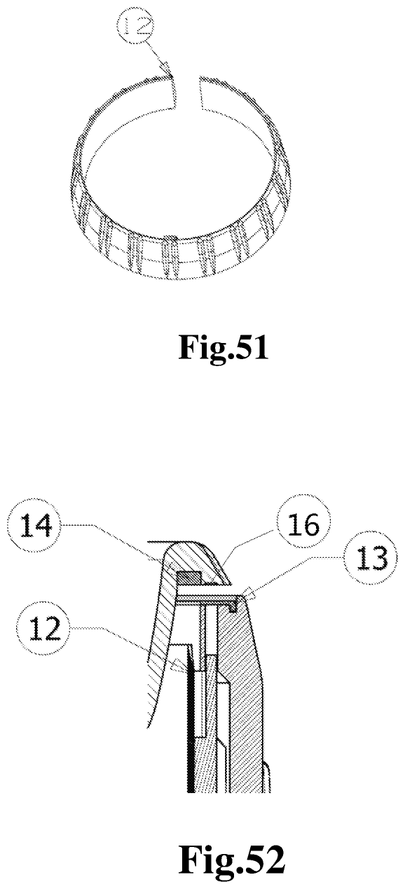

FIGS. 40-43 are schematic diagrams of structural positions of the indication device pre-disposed on the comprehensive cover, which may be a protrusion 1, a groove 2, a convex point 3 or a line 4, are directly designed as a concave or convex structure on the top end of the comprehensive cover and strictly correspond to the U-nail-related structures inside. FIGS. 44-47 are schematic structural diagrams of the prefabricated indication device in a ferrule form, which may be a protrusion ferrule 5, a groove ferrule 6, a convex point ferrule 7 or a line ferrule 8. FIGS. 48-51 are schematic structural diagrams of the prefabricated indication device in a sticky label form, which may be a protrusion sticky label 9, a groove sticky label 10, a convex point sticky label 11 or a line sticky label 12 and applied to a prepuce circumcision stapler that is not provided with a blood vessel indication device, wherein the indication devices in the ferrule form have different inner diameters and can adapt to the comprehensive cover or other parts of different kinds of prepuce circumcision staplers, thereby corresponding to the U-nail-related structures inside. The indication devices in the sticky label form comprise different specifications and can adapt to the comprehensive covers or other parts of different kinds of prepuce circumcision staplers, thereby corresponding to the U-nail-related structures inside, wherein the front surface of the indication device is of a concave or convex structure, and the back surface of the indication device is an adhesive layer or release paper.

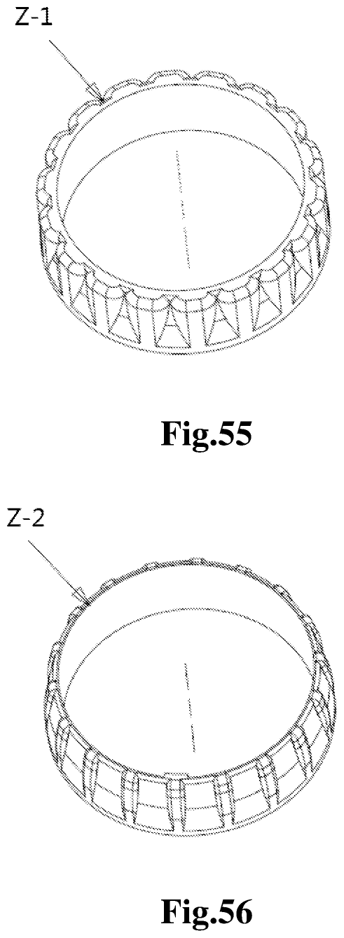

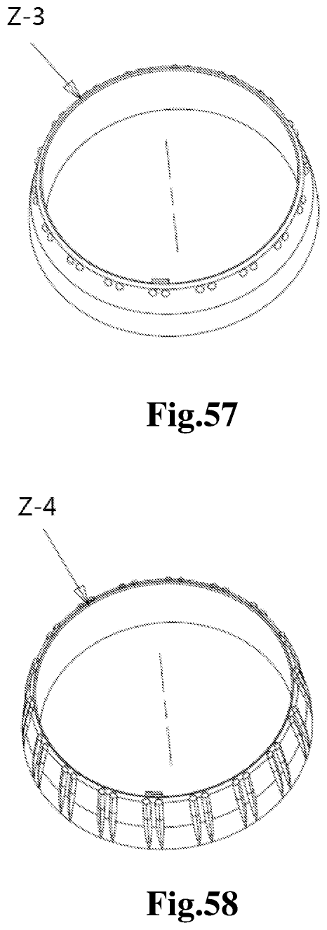

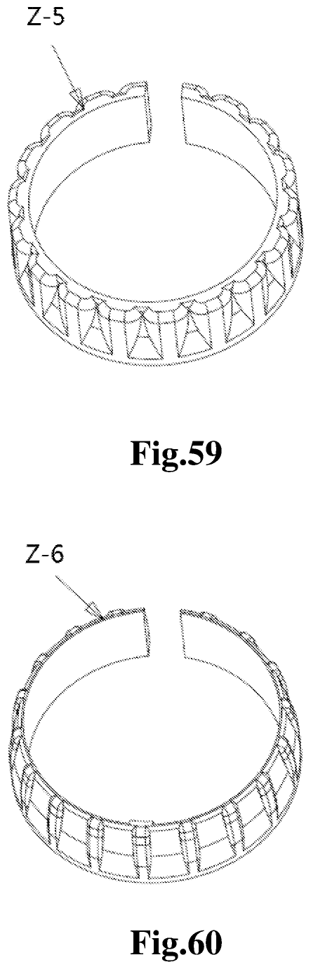

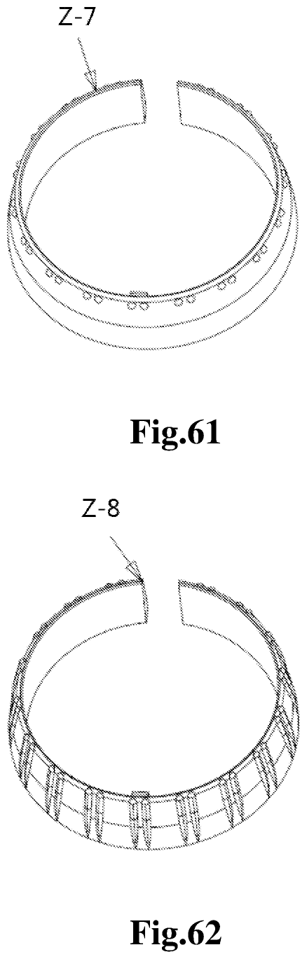

Regarding the valgus type, FIGS. 55-58 are schematic structural diagrams of the prefabricated indication device in a ferrule form, which may be a protrusion ferrule Z-1, a groove ferrule Z-2, a convex point ferrule Z-3 or a line ferrule Z-4. FIGS. 59-62 are schematic structural diagrams of the prefabricated indication device in a sticky label form, which may be a protrusion sticky label Z-5, a groove sticky label Z-6, a convex point sticky label Z-7 or a line sticky label Z-8 and applied to a prepuce circumcision stapler that is not provided with a blood vessel indication device, wherein the indication devices in the ferrule form have different inner diameters and can adapt to the comprehensive cover or other parts of different kinds of prepuce circumcision staplers, thereby corresponding to the U-nail-related structures inside. The indication devices in the sticky label comprise different specifications and can adapt to the comprehensive cover or other parts of different kinds of prepuce circumcision staplers, wherein the front surface of the indication device is of a concave or convex structure, and the back surface of the indication device is an adhesive layer or release paper.

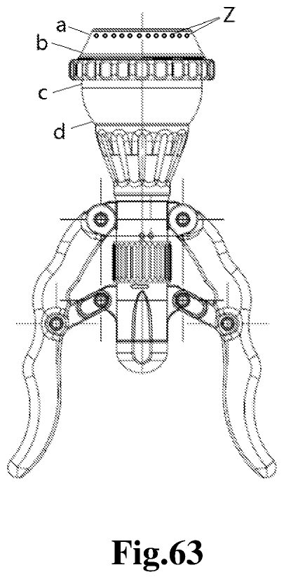

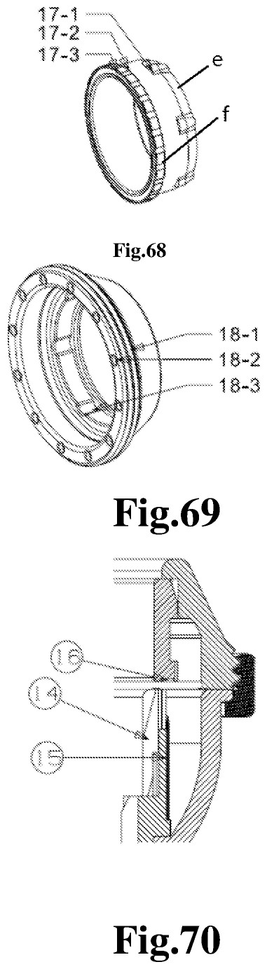

FIG. 63 is an overall view of the valgus prepuce circumcision stapler of the present invention, in which positions indicated by a, b, c and d are preferred positions for arrangement of the indication device. Exemplarily, a ring of convex indication points Z is arranged on the position a. FIG. 68 is the enlarged view of the inner ring, in which positions indicated by e and f are preferred positions for arrangement of the indication device, wherein the upper end or the lower end of the outer surface of the inner ring may be provided with a graduation device, a concave or convex touch sensing device or a blood vessel docking groove structure according to specific demands.

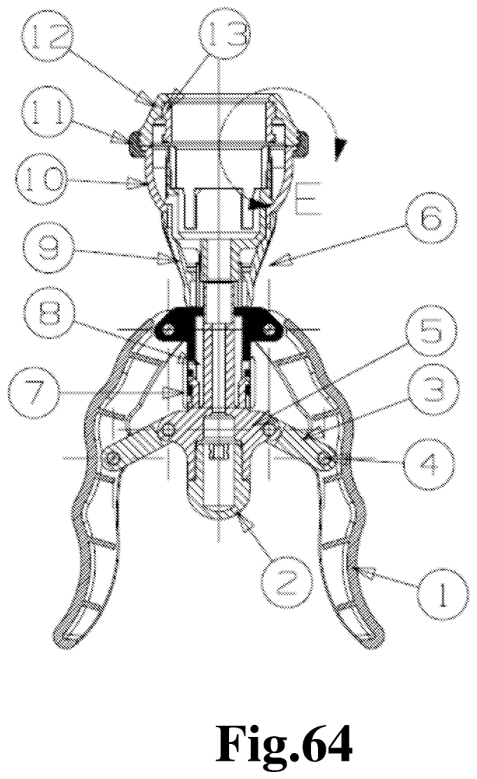

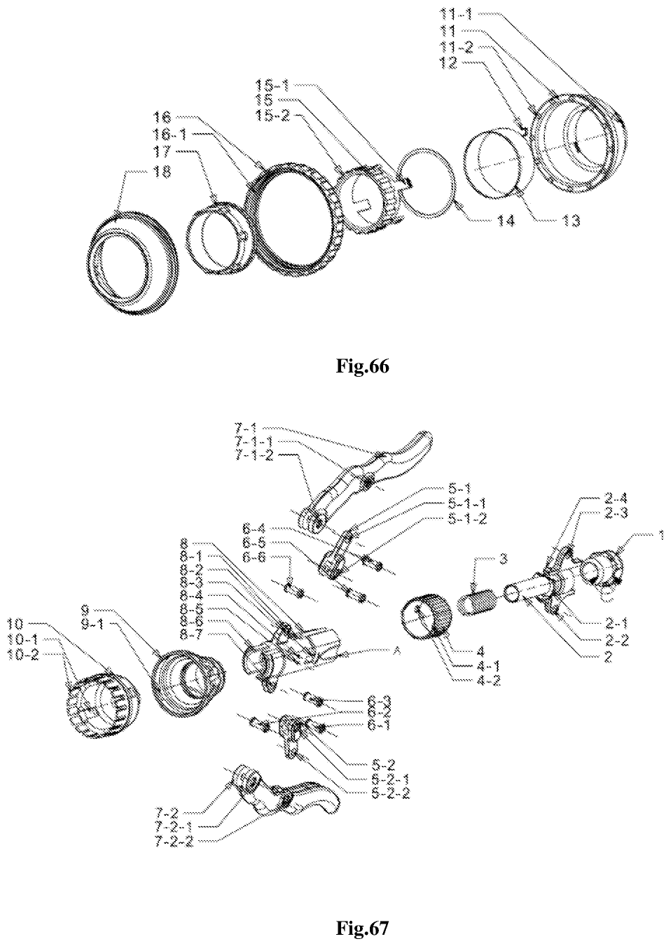

FIGS. 64-70 are structure views of the valgus prepuce circumcision stapler of the present embodiment. In the drawings, reference signs represent the following components: handles 1; plug 2; small connecting rod 3; pin 4; sliding rod 5; a connecting block 6; an opening knob 7; spring 8; comprehensive cover 9; lower cover 10; locking ring 11; upper cover 12; inner ring 13; inner comprehensive cover 14; annular blade 15; first washer/U-nail backing ring 16. The handles 1 are connected to the sliding rod 5 through the small connecting rods 3 or the pins 4 to form a driving component. The opening knob 7 sleeves the periphery of the connecting block and serves to open or lock the handles, such that the handles can be or cannot be driven. The lower end of the comprehensive cover 9 is connected to the connecting block. The upper end of the comprehensive cover 9 is connected to the lower end of the lower cover 10. The upper end of the lower cover 10 is connected to the lower end of the upper cover 12 through the locking ring 11. The inner ring 13 is fully or partially arranged in the upper cover 12. The inner comprehensive cover 14 is fully or partially arranged in the lower cover 10. The inner ring 13 and the inner comprehensive cover 14 serve to cooperate with each other and clamp the prepuce, or the inner ring 13 and the upper cover 12 serves to cooperate with each other and clamp the prepuce. The annular blade 15 is arranged on the periphery of the inner comprehensive cover 14 and is driven to make a circumcision on the prepuce. The U-nail backing ring 16 is arranged at the lower end of the inner ring and correspondingly allows the U-nails to penetrate through. With reference to the drawing, the position relationships and connection relationships of other members are both clear, and the details will not be repeated here.

FIGS. 65-67 and 46 are exploded views of a valgus prepuce circumcision stapler. The members includes a plug 1, a positioning concave point 1-1, a sliding rod 2, a spring supporting platform 2-1, a pin hole 2-2, a pin hole 2-3, a hook 2-4, a spring 3, an opening knob 4, a pointer 4-1, a sliding block 4-2, a connecting rod 5, a small connecting rod 5-1, a pin hole 5-1-1, a pin hole 5-1-2, a small connecting rod 5-2, a pin hole 5-2-1, a pin hole 5-2-2, a pin 6-1, a pin 6-2, a pin 6-3, a pin 6-4, a pin 6-5, a pin 6-6, a handle 7-1, a pin hole 7-1-1, a pin hole 7-1-2, a handle 7-2, a pin hole 7-2-1, a pin hole 7-2-2, a connecting block 8, a clamping platform 8-1, a pin hole 8-2, an opening guide groove 8-3, a closing mark 8-4, an opening mark 8-5, a hook 8-6, a pin hole 8-7, a positioning convex point 8-8, a comprehensive cover 9, a clamping groove 9-1, a U-nail top ring 10, a groove 10-1, a U-nail top column 10-2, a lower cover 11, a clamping platform 11-1, a positioning platform 11-2, a U-nail 12, a circumcision knife 13, a U-nail backing ring 14, an inner comprehensive cover 15, a hook 15-1, U-nail grooves 15-2, a locking ring 16, a thread 16-1, an inner ring 17, positioning platforms 17-1, blood vessel docking grooves 17-2, a U-nail ring pad groove 17-3, an upper cover 18, a thread 18-1, positioning grooves 18-2, and positioning groove 18-3. The inner ring 17 and the inner comprehensive cover 15 serve to cooperate with each other and clamp the prepuce, or the inner ring 17 and the upper cover 18 serve to cooperate with each other and clamp the prepuce. The U-nail top ring 10 is assembled to and cooperates with the inner comprehensive cover 15, thereby pushing the U-nails 12 out from the U-nail grooves 15-2. The annular knife 13 is arranged on the periphery of the inner comprehensive cover and is driven by the driving component to make a circumcision on the prepuce. The upper cover 18 and the lower cover 11 are assembled to and connected with each other. The inner ring 17 is arranged in the upper cover 18. The inner comprehensive cover 15 is fully or partially arranged in the lower cover 11. The front end of the comprehensive cover 9 is connected to the rear end of the lower cover 11. The U-nail top ring 10 is partially arranged in the comprehensive cover 9. The inner comprehensive cover 15 is provided with a plurality of U-nail grooves 15-2 for accommodating the U-nails 12. The U-nail grooves 15-2 are arranged along the periphery of the front end of the inner comprehensive cover 15. A nail outlet of the U-nail groove 15-2 is arranged close to the top end of the inner comprehensive cover 15. The upper cover 18 and the lower cover 11 are tightened and cooperate through the locking ring 16. The upper end of the connecting block 8 is connected to the comprehensive cover 9. The lower end of the connecting block 8 is connected to the sliding rod 2 and the plug 1. The opening knob which can open or close a driving device, such as the handles, is arranged outside the connecting block 8. A plurality of vessel docking grooves 17-2 is arranged along the periphery of the rear end of the inner ring 17. The positions of the plurality of blood vessel docking grooves 17-2 are in one-to-one correspondence to those of the U-nail grooves 15-2. The blood vessel docking grooves 17-2 are pre-disposed on the inner ring 17. The rear ends of the blood vessel docking grooves 17-2 are flush with the rear end of the inner ring 17. The front ends of the blood vessel docking grooves 17-2 are away from the annular rear end of the inner ring 17 forwards relative to the rear ends of the blood vessel docking grooves 17-2. The front end of the inner ring 17 is provided with positioning platforms 17-1 for positioning. The periphery of the rear end of the inner ring 17 is provided with the blood vessel docking grooves 17-2. The rear end surface of the inner ring 17 is provided with a U-nail ring pad groove 17-3 for corresponding to the U-nails. The outer periphery of the rear end of the upper cover 18 is provided with the thread 18-1 and the positioning grooves 18-2 for connecting with the lower cover 11 and the locking ring 16. The central part close to the front end of the upper cover 18 is provided with positioning grooves 18-3 for corresponding to the positioning platforms 17-1. With reference to the drawing, the position relationships and connection relationships of other structures are both clear, and the details will not be repeated here.

In a preferred embodiment, the valgus prepuce circumcision stapler with an indication device comprises an upper cover 18, a lower cover 11, an inner ring 17, an inner comprehensive cover 15 and a locking ring 16, and the indication device. The upper cover 18 and the lower cover 11 are assembled to and cooperate with each other upwards and downwards. The inner ring 17 is arranged in the upper cover 18. The inner comprehensive cover 15 is fully or partially arranged in the lower cover 11. The indication device is arranged on the inner ring 17 or the inner comprehensive cover 15. The locking ring 16 is used for locking the upper cover 18 and the lower cover 11. The indication device Z is a ring of protrusions which are annularly arranged. The protrusion degree of the protrusions can be contacted and perceived by an operator when the prepuce covers the protrusions.

A plurality of U-nail grooves 15-2 which is annularly arranged and constitutes a ring is arranged on the periphery of the inner comprehensive cover 15. The protrusions or recesses of the indication device are arranged to be in one-to-one correspondence with the U-nail grooves 15-2 and/or intervals among the U-nail grooves 15-2 and/or the U-nails in the U-nail grooves 15-2; or, intervals among the protrusions or recesses of the indication device are in one-to-one correspondence with the U-nail grooves 15-2 and/or the intervals among the U-nail grooves 15-2 and/or the U-nails in the U-nail grooves 15-2. The protrusions or recesses of the indication device are arranged such that the width between the two protrusions or recesses is equal to the width of the U-nail groove 15-2, and/or the width of the U-nail in the U-nail groove 15-2, and/or the width of the interval between the U-nail grooves 15-2.

In a preferred embodiment, the indication device is protrusions or recesses which are prefabricated on the upper cover 18, and/or the lower cover 11, and/or the inner ring 17, and/or the inner comprehensive cover 15, and/or the locking ring 16; or an indication label (Z-5, Z-6, Z-7 or Z-8) which is adhered to the upper cover 18, and/or the lower cover 11, and/or the inner ring 17, and/or the inner comprehensive cover 15, and/or the locking ring 16 and has protrusions or recesses; or a prefabricated indication ring or indication strip (Z-1, Z-2, Z-3 or Z-4) which sleeves the upper cover 18, and/or the lower cover 11, and/or the inner ring 17, and/or the inner comprehensive cover 15, and/or the locking ring 16 and has protrusions or recesses.

In a preferred embodiments, the indication device is graduations prefabricated on the periphery of the upper end and/or the periphery of the lower end of the inner ring 17, and/or a graduation label adhered to the periphery of the upper end and/or the periphery of the lower end of the inner ring 17, and/or a prefabricated graduation ring or graduation strip which sleeves the periphery of the upper end and/or the periphery of the lower end of the inner ring 17, and/or blood vessel docking grooves 17-2 which are prefabricated on the periphery of the upper end and/or the periphery of the lower end of the inner ring 17.

In a preferred embodiment, the indication device is graduations prefabricated on the periphery of the upper end and/or the periphery of the lower end of the inner comprehensive cover 15, and/or a graduation label adhered to the periphery of the upper end and/or the periphery of the lower end of the inner comprehensive cover 15, and/or a prefabricated graduation ring or graduation strip which sleeves the periphery of the upper end and/or the periphery of the lower end of the inner comprehensive cover 15, and/or a blood vessel docking groove 17-2 which is prefabricated on the periphery of the upper end and/or the periphery of the lower end of the inner comprehensive cover 15.

The protrusions are convex blocks and/or convex points and/or convex strips, and the recesses are grooves and/or concave points and/or concave strips. Both the left side surface and the right side surface and/or both the upper end surface and the lower end surface of the convex block have one or a plurality of oblique surfaces; and/or, one or two of the convex points constitute a group and are circular convex points; and/or one or two of the convex strips constitute a group, and both the left side surface and the right side surface of each convex strip have one or a plurality of oblique surfaces; and/or, both the left side surface and the right side surface and/or both the upper end surface and the lower end surface of the groove have one or a plurality of oblique surfaces; and/or, one or two of the concave points constitute a group and are circular concave points; and/or, one or two of the concave strips constitute a group, and both the left side surface and the right side surface of each concave strip have one or a plurality of oblique surfaces; and/or, the convex strip is a vertical convex strip, and the upper end and the lower end of the convex strip are curved; and/or, the concave strip is a vertical concave strip, and the upper end and the lower end of the convex strip are curved.

The prepuce circumcision stapler further comprises a comprehensive cover 9, wherein the upper end of the comprehensive cover 9 cooperates with and is connected to the lower end of the lower cover 11. The indication device is arranged at one end, close to the upper cover 18, of the outer surface of the lower cover 11; and/or, the indication device is arranged at one end, close to the comprehensive cover, of the outer surface of the lower cover 11; and/or, the indication device is arranged at one end, close to the lower cover 11, of the outer surface of the upper cover 18; and/or, the indication device is arranged at the upper end of the outer surface of the upper over 18; and/or, the indication device is arranged at the upper end of the outer surface of the inner ring 17; and/or, the indication device is arranged at the lower end of the outer surface of the inner ring 17; and/or, the indication device is arranged at the upper end of the outer surface of the inner comprehensive cover 15; and/or, the indication device is arranged at the lower end of the outer surface of the inner comprehensive cover 15; and/or, the indication device is arranged on the periphery of the locking ring 16.

In another preferred embodiment in which the structures shown in FIGS. 65-67 and 46 are adopted, a valgus prepuce circumcision stapler with an indication device comprises an upper cover, and/or a lower cover, and/or an inner ring, and/or an inner comprehensive cover, and further comprises the indication device. The upper cover and the lower cover are assembled to and cooperate with each other upwards and downwards. The inner ring is arranged in the upper cover. The inner comprehensive cover is fully or partially arranged in the lower cover. The indication device is arranged on the upper cover, and/or the lower cover, and/or the inner ring, and/or the inner comprehensive cover.

The valgus prepuce circumcision stapler further comprises a locking ring which is used for locking the upper cover and the lower cover and is provided with the indication device. The indication device is a ring of protrusions or recesses which are annularly arranged. Preferably, the concave or convex degree of the protrusions or recesses allows that the protrusions or recesses can be contacted and perceived by an operator when the prepuce covers or does not cover the protrusions or recesses. A plurality of U-nail grooves which constitutes a ring is annularly arranged on the periphery of the inner comprehensive cover. The protrusions or recesses of the indication device are arranged to be in one-to-one correspondence with the U-nail grooves and/or intervals among the U-nail grooves and/or the U-nails in the U-nail grooves; or, intervals among the protrusions or recesses of the indication device are in one-to-one correspondence with the U-nail grooves and/or the intervals among the U-nail grooves and/or the U-nails in the U-nail grooves. The protrusions or recesses of the indication device are arranged such that the width between the two protrusions or recesses is equal to the width of the U-nail groove, and/or the width of the U-nail in the U-nail groove, and/or the width of the interval between the U-nail grooves.

The indication device is protrusions or recesses which are prefabricated on the upper cover, and/or the lower cover, and/or the inner ring, and/or the inner comprehensive cover, and/or the locking ring, and/or an indication label which is adhered to the upper cover, and/or the lower cover, and/or the inner ring, and/or the inner comprehensive cover, and/or the locking ring and has protrusions or recesses, and/or a prefabricated indication ring or strip which sleeves the upper cover, and/or the lower cover, and/or the inner ring, and/or the inner comprehensive cover, and/or the locking ring and has protrusions or recesses; or,
the indication device is graduations prefabricated on the periphery of the upper end and/or the periphery of the lower end of the inner ring, and/or a graduation label adhered to the periphery of the upper end and/or the periphery of the lower end of the inner ring, and/or a prefabricated graduation ring or graduation strip which sleeves the periphery of the upper end and/or the periphery of the lower end of the inner ring, and/or blood vessel docking grooves which are prefabricated on the periphery of the upper end and/or the periphery of the lower end of the inner ring; or,
the indication device is graduations prefabricated on the periphery of the upper end and/or the periphery of the lower end of the inner comprehensive cover, and/or a graduation label adhered to the periphery of the upper end and/or the periphery of the lower end of the inner comprehensive cover, and/or a prefabricated graduation ring or graduation strip which sleeves the periphery of the upper end and/or the periphery of the lower end of the inner comprehensive cover, and/or blood vessel docking grooves which are prefabricated on the periphery of the upper end and/or the periphery of the lower end of the inner comprehensive cover.

The protrusions are convex blocks and/or convex points and/or convex strips, and the recesses are grooves and/or concave points and/or concave strips. Both the left side surface and the right side surface and/or both the upper end surface and the lower end surface of the convex block have one or a plurality of oblique surfaces; and/or, one or two of the convex points constitute a group and are circular convex points; and/or one or two of the convex strips constitute a group, and both the left side surface and the right side surface of each convex strip have one or a plurality of oblique surfaces; and/or, both the left side surface and the right side surface and/or both the upper end surface and the lower end surface of the groove have one or a plurality of oblique surfaces; and/or, one or two of the concave points constitute a group and are circular concave points; and/or, one or two of the concave strips constitute a group, and both the left side surface and the right side surface of each concave strip have one or a plurality of oblique surfaces; and/or, the convex strip is a vertical convex strip, and the upper end and the lower end of the convex strip are curved; and/or, the concave strip is a vertical concave strip, and the upper end and the lower end of the convex strip are curved.

The prepuce circumcision stapler further comprises a comprehensive cover, wherein the upper end of the comprehensive cover cooperates with and is connected to the lower end of the lower cover. The indication device is arranged at one end, close to the upper cover, of the outer surface of the lower cover; and/or, the indication device is arranged at one end, close to the comprehensive cover, of the outer surface of the lower cover; and/or, the indication device is arranged at one end, close to the lower cover, of the outer surface of the upper cover; and/or, the indication device is arranged at the upper end of the outer surface of the upper over; and/or, the indication device is arranged at the upper end of the outer surface of the inner ring; and/or, the indication device is arranged at the lower end of the outer surface of the inner ring; and/or, the indication device is arranged at the upper end of the outer surface of the inner comprehensive cover; and/or, the indication device is arranged at the lower end of the outer surface of the inner comprehensive cover; and/or, the indication device is arranged on the periphery of the locking ring.

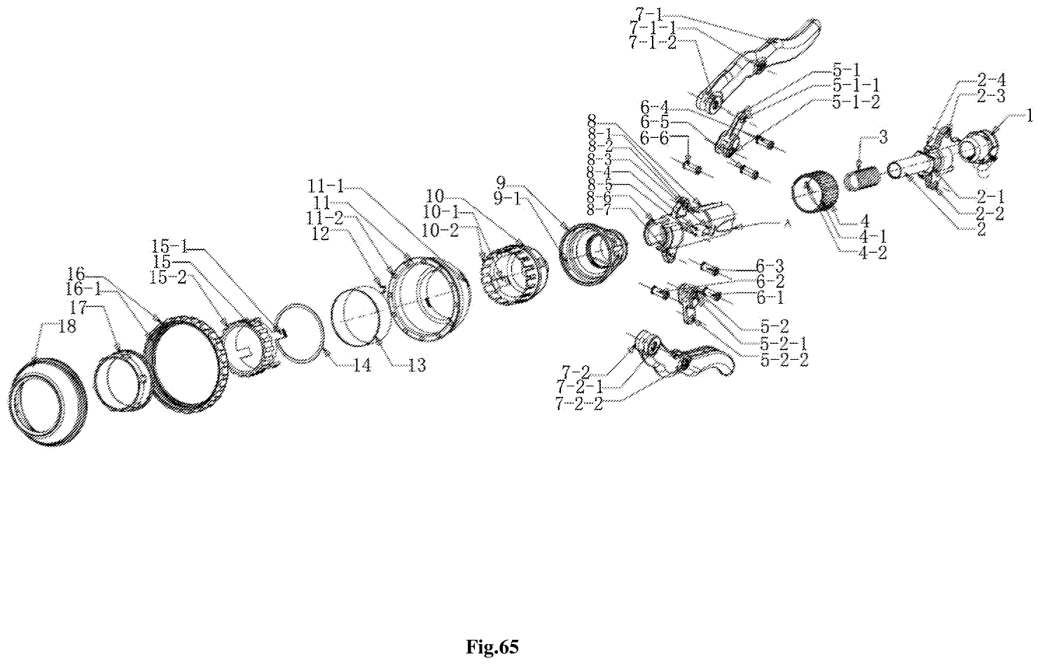

In another preferred embodiment shown in FIGS. 65-69, a valgus prepuce circumcision stapler comprises an upper cover 18, a lower cover 11, an inner ring 17 and an inner comprehensive cover 15. The upper cover 18 and the lower cover 11 may be removably cooperates with and is connected to the upper cover 18 and is provided with a first through hole in the middle. The upper cover 18 has a cavity for accommodating and positioning the inner ring 17. The lower cover 11 is provided with a third through hole in the middle and has a cavity for accommodating and positioning the inner comprehensive cover 15. The inner ring 17 is fully or partially arranged in the upper cover 18. The inner ring 17 is a circular ring provided with a second through hole in the middle. The second through hole corresponds to the first through hole. The inner comprehensive cover 15 is fully or partially arranged in the lower cover 11. The inner comprehensive cover 15 is a circular ring provided with a fourth through hole in the middle. The fourth through hole corresponds to the second through hole. The inner ring 17 and the inner comprehensive cover 15 serve to cooperate with each other upwards and downwards to clamp the prepuce, or the inner ring 17 and the upper cover 18 serve to cooperate with each other to clamp the prepuce.

The prepuce circumcision stapler further comprises a U-nail top ring 10, a circumcision knife 13 and a locking ring 16. The inner ring surface of the locking ring 16 is provided with an inner thread 16-1. The outer surface of the lower end of the upper cover 18 is provided with an outer thread 18-1. The inner thread 16-1 and the outer thread 18-1 may cooperate with each other to position the upper cover 18, the lower cover 11 and the locking ring 16. The circumcision knife 13 sleeves the periphery of the inner comprehensive cover 15 and is fully or partially arranged in the lower cover 11. The upper end of the U-nail top ring 10 is provided with U-nail top columns 10-2 which are in one-to-one correspondence to the U-nail grooves 15-2. The U-nail top columns 10-2 are used for pushing the U-nails 12 out from the U-nail grooves 15-2 when moving upwards. A cavity of the U-nail top ring 10 is provided with grooves 10-1 which are in one-to-one correspondence to the convex strips and hooks 15-1. The grooves 10-1 serve to position the convex strips and the hooks 15-1 in a manner of cooperating with each other.

The lower end surface of the upper cover 18 is an annular end surface which is provided with concave positioning grooves 18-2. The upper end surface of the lower cover 11 is an annular upper end surface which is provided with convex positioning platforms 11-2. The positioning grooves 18-2 and the positioning platforms 11-2 cooperate with each other in a concave or convex manner to position the upper cover 18 and the lower cover 11. The cavity of the upper cover 18 is provided with a plurality of positioning grooves 18-3. The outer surface of the inner ring 17 is provided with a plurality of positioning platforms 17-1. The positioning grooves 18-3 and the positioning platforms 17-1 cooperate with each other in a concave or convex manner to position the upper cover 18 and the inner ring 17. The outer surface of the inner comprehensive cover 15 is provided with a ring of U-nail grooves 15-2. The U-nail grooves 15-2 are used for accommodating the U-nails 12 for hemostasis of a prepuce circumcision surface. The lower end of the inner comprehensive cover 15 is provided with convex strips and hooks 15-1.

The prepuce circumcision stapler further comprises a U-nail backing ring 14, a comprehensive cover 9, a connecting block 8, a sliding rod 2, a plug 1 and handles 7-1/7-2. The U-nail backing ring 14 is annular backing ring and is arranged in the U-nail ring pad groove 17-3 at the lower end, corresponding to the top end of the inner comprehensive cover 15, of the inner ring 17. The upper end of the comprehensive cover 9 is connected to the lower end of the lower cover 11. A cavity in the comprehensive cover 9 is used for accommodating all of or a part of the U-nail top columns 10-2. The inner wall of the comprehensive cover 9 is provided with a clamping groove 9-1. The lower end of the lower cover 11 is provided with a clamping platform 11-1. The clamping groove 9-1 and the clamping platform 11-1 cooperate with and are fixedly connected to each other. The upper end of the connecting block 8 is connected to the lower end of the comprehensive cover 9. The upper end of the sliding rod 2 is inserted to the lower end of the connecting block 8. The plug 1 is arranged at the lower end of the sliding rod 2. The handles 7-1/7-2 can be driven to be connected to the connecting block 8 and serves to drive the sliding rod 2 and a driving component inside the connecting block, thereby driving the U-nail top ring 10 and the circumcision knife 13 to move upwards. The outer surface of the lower end of the inner ring 17 is provided with a ring of groove structures for docking with blood vessels. With reference to the drawing, the position relationships and connection relationships of related driving components, actuating components and circumcision components are both clear, and the details will not be repeated here.

In another preferred embodiment, the blood vessel docking device of the valgus prepuce circumcision stapler includes a comprehensive cover 9, a U-nail top ring 10, a lower cover 11, an inner comprehensive cover 15, an inner ring 17, an upper cover 18 and a blood vessel docking groove 17-2. The inner ring 17 and the inner comprehensive cover 15 are used to cooperate with each other. A plurality of blood vessel docking grooves 17-2 are provided along the periphery of a rear end of the inner ring 17. The U-nail top ring 10 is used for being assembled to and cooperating with the inner comprehensive cover 15 to push a U-nail 12 out of a U-nail groove 15-2. The upper cover 18 and the lower cover 11 are used for being assembled and connected to each other. The inner ring 17 is arranged in the upper cover 18. The inner comprehensive cover 15 is partially arranged in the lower cover 11. A front end of the comprehensive cover 9 is connected to a rear end of the lower cover 11. The U-nail top ring 10 is partially arranged in the comprehensive cover 9.

The inner comprehensive cover 15 is provided with a plurality of U-nail grooves 15-2 for accommodating the U-nails 12. The U-nail grooves 15-2 are arranged along the periphery of a front end of the inner comprehensive cover 15. A nail outlet of the U-nail groove 15-2 is provided close to a top end of the inner comprehensive cover 15. The positions of the plurality of blood vessel docking grooves 17-2 correspond to the positions of the U-nail grooves 15-2 one by one. The blood vessel docking groove 17-2 is preset on the inner ring 17. A rear end of the blood vessel docking groove 17-2 is flush with the rear end of the inner ring 17. A front end of the blood vessel docking groove 17-2 is away from the annular rear end of the inner ring 17 with respect to the rear end of the blood vessel docking groove 17-2.

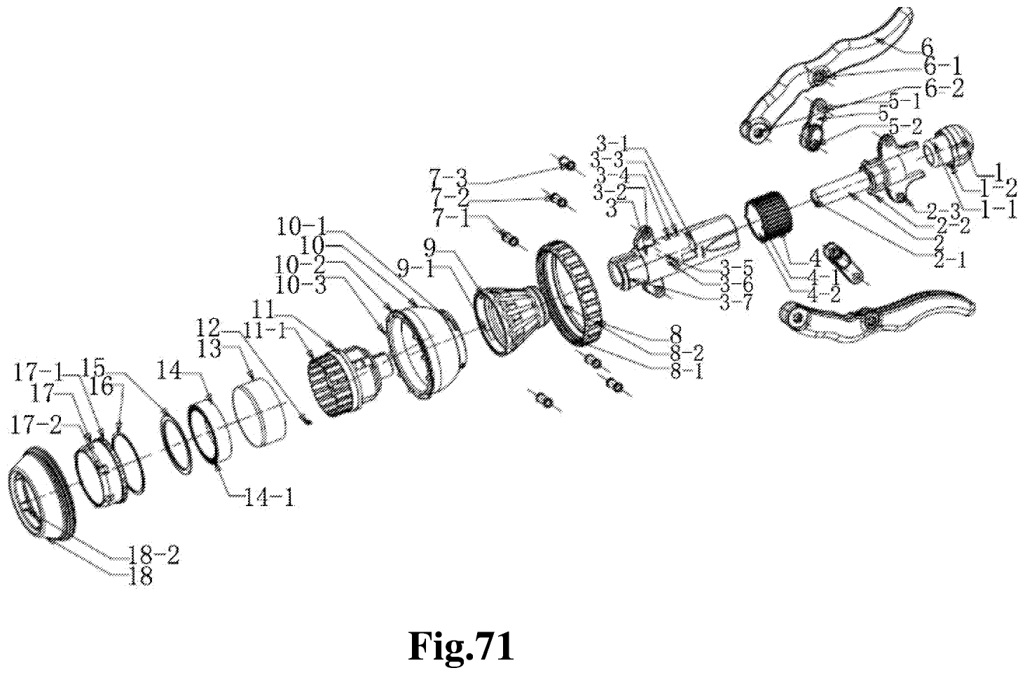

FIG. 71 is an exploded view of a valgus prepuce circumcision stapler, which includes a plug 1, a round table 1-1, a groove 1-2, a push rod 2, a boss 2-1, a first hook 2-2, a first pin hole 2-3, a connecting block 3, a guide groove 3-1, a second pin hole 3-2, a first hanging table 3-3, a second hanging table 3-4, a close indicator 3-5, an open indicator 3-6, a second hook 3-7, an open knob 4, a pointer 4-1, a middle circular hole 4-2, a guide block 4-3, a small link 5, a third pin hole 5-1, a fourth pin hole 5-2, a handle 6, a fifth pin hole 6-1, a sixth pin hole 6-2, a first pin 7-1, a second pin 7-2, a third pin 7-3, a locking ring 8, a thread 8-1, a collar 8-2, a lower comprehensive cover 9, a hook 9-1, a connecting cover 10, a hook 10-1, a collar 10-2, a positioning boss 10-3, a U-nail top ring 11, a U-nail 12, a ring cutter 13, a thimble guide block 14, a thimble guide groove 14-1, a U-nail spacer 15, a spacer positioning table 15-1, a blade washer 16, a prepuce fixing hoop 17, a blood vessel docking groove 17-1, a positioning table 17-2, an upper comprehensive cover 18 and a positioning groove 18-2. The valgus prepuce circumcision stapler includes an upper comprehensive cover 18, a lower comprehensive cover 9, a prepuce fixing hoop 17 and a U-nail device. The upper comprehensive cover 18 is detachably cooperating and connected with the lower comprehensive cover 9. The prepuce fixing hoop 17 is arranged in the upper comprehensive cover 18. The U-nail device is arranged in the lower comprehensive cover 9. The prepuce fixing hoop 17 can be positioned within the upper comprehensive cover 18 and cooperating with the upper comprehensive cover 18 to clamp a prepuce. A lower end of the prepuce fixing hoop 17 can be cooperating with the U-nail device to clamp the prepuce.

The U-nail device includes a U-nail top ring 11 and a thimble guide block 14. The thimble guide block 14 is used for accommodating a suturing U-nail 12, guiding the movement of the suturing U-nail 12, and positioning and guiding a thimble of the U-nail top ring 11. The U-nail top ring 11 is used to cooperate with the thimble guide block 14 to push out the U-nail 12 out. Also included is a U-nail spacer 15, which is an annular spacer arranged on the top end of the thimble guide block 14. The U-nail spacer 15 is also provided with a spacer positioning table 15-1 perpendicular to the spacer plane. The spacer positioning table 15-1 is a complete annular protrusion arranged on the inner periphery or outer periphery or middle of the spacer plane of the U-nail spacer 15. A first through hole is formed in the middle of the thimble guide block 14. The thimble guide block 14 includes an annular inner wall, an annular outer wall, and a positioning platform accommodating groove. The annular inner wall is used for cooperating with an annular protrusion arranged on the inner periphery of the spacer plane of the U-nail spacer 15 for positioning. The annular outer wall is used for cooperating with an annular protrusion arranged on the outer periphery of the spacer plane of the U-nail spacer 15 for positioning. The positioning platform accommodating groove is arranged at the top end of the thimble guide block 14 and is used to accommodate an annular protrusion in the middle of the spacer plane of the U-nail spacer 15. The thimble guide block 14 is provided with a plurality of thimble guide grooves 14-1. The thimble guide grooves 14-1 are annularly arranged on the annular thimble guide block 14. The U-nail spacer 15 completely covers the thimble guide groove 14-1.

Also included is a connecting cover 10, which is arranged between the upper comprehensive cover 18 and the lower comprehensive cover 9. The upper comprehensive cover 18 is detachably connected to the lower comprehensive cover 9 through the connecting cover 10. A lower end face of the upper comprehensive cover 18 is annular, along which a concave positioning groove is provided. An upper end face of the connecting cover 10 is annular, along which a convex positioning boss 10-3 is provided. The positioning groove and the positioning boss 10-3 cooperate with each other in a concave and convex manner to position the upper comprehensive cover 18 and the connection cover 10. The upper end face of the connecting cover 10 is annular, along which a convex positioning boss 10-3 is provided. The positioning boss 10-3 and the positioning groove arranged on the lower end face of the upper comprehensive cover 18 cooperate with each other for positioning. A plurality of hooks 10-1 are arranged at a lower part of the connecting cover 10. A plurality of hooks 9-1 are arranged at an upper part of the lower comprehensive cover 9. The hooks between the connecting cover 10 and the lower comprehensive cover 9 cooperate with each other for positioning.

Also included is a locking ring 8. An inner surface of the locking ring 8 is provided with an internal thread 8-1 and/or a collar 8-2. An external thread is arranged on an outer surface of the lower end of the upper cover 18. An outer surface of the upper end of the connecting cover 10 is provided with an external thread or collar 10-2. The upper comprehensive cover 18, the connecting cover 10, the lower comprehensive cover 9, and the locking ring 8 can be positioned by means of the cooperation between the internal thread 8-1 and the external thread, and/or the collar 8-2/10-2.

The middle of the upper comprehensive cover 18 is provided with a second through hole. The upper comprehensive cover 18 has an inner cavity for receiving and positioning the prepuce fixing hoop 17. The prepuce fixing hoop 17 is a ring provided with a third through hole in the middle thereof. The third through hole corresponds to the second through hole. The inner cavity of the upper comprehensive cover 18 is provided with a plurality of positioning grooves. An outer surface of the prepuce fixing hoop 17 is provided with a plurality of positioning tables 17-2. Positioning grooves 18-2 and the positioning tables 17-2 cooperate with each other for positioning. Also included is a ring cutter 13. The ring cutter 13 is sleeved around the thimble guide block 14 and U-nail top ring 11. The ring cutter 13 can be driven by the U-nail top ring 11 to move. Also included is a blade washer 16 which is arranged at a lower end face of the prepuce fixing hoop 17 and corresponds to the position of the ring cutter 13. The U-nail device is partially arranged in the lower comprehensive cover 9 and partially arranged in the connecting cover 10. Also included is a connecting block 3, an upper end of which is connected to a lower end of the lower comprehensive cover 9. Also included is a push rod, an upper end of which is inserted into a lower end of the connecting block 3. Also included is a plug 1 which is arranged at a lower end of the push rod. Also included is a handle 6 which is drivingly connected to the connecting block 3 and push rod 2, and used to drive a transmission member of the push rod 2 and the connecting block 3 so as to drive the U-nail top ring 11 and the ring cutter 13 to move upward. An outer surface of the lower end of the prepuce fixing hoop is provided with a ring of blood vessel docking grooves 17-1 for blood vessel docking. Also included is an open knob 4 which is sleeved on the connecting block 3 for locking or unlocking the handle and/or a component driven by the handle. FIGS. 75 and 76 are an enlarged view of the U-nail spacer and the open knob respectively. An upper end of the open knob 4 is provided with a pointer 4-1. The middle thereof is provided with a circular hole 4-2. A guide block 4-3 is arranged on an inner wall to cooperate with a guide groove 3-1 arranged on the connecting block 3 so as to lock or unlock the handle or other driving member. One side of the U-nail spacer 15 is provided with a spacer positioning table 15-1 for positioning it on the thimble guide block 14.

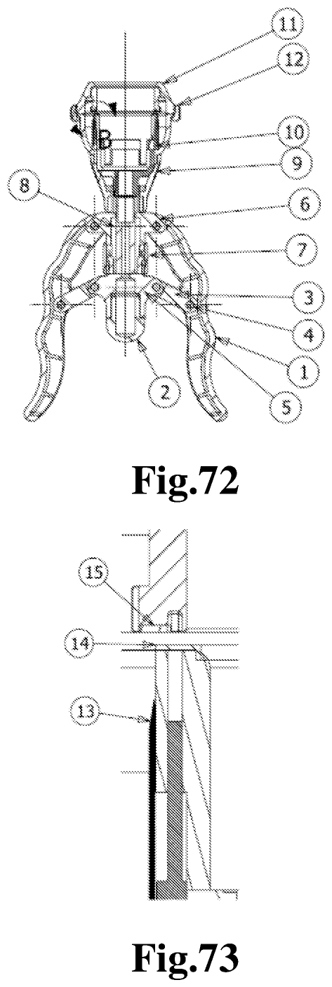

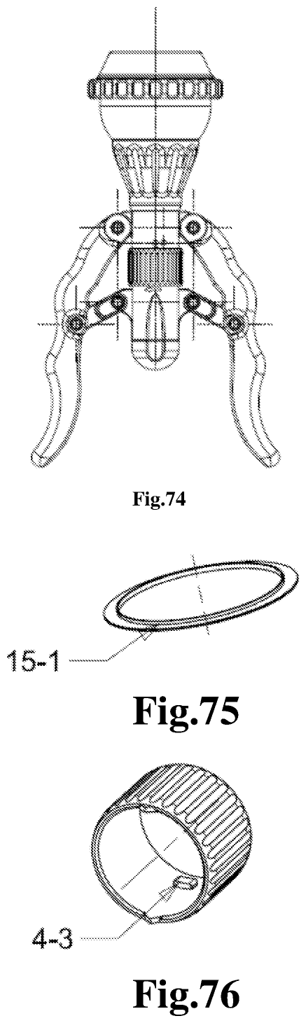

FIG. 74 is a front view of the valgus prepuce cutting stapler. Referring to FIGS. 72 and 73, in the preferred embodiment, the valgus prepuce cutting stapler includes a handle 1, a plug 2, a small link 3, a pin 4, a push rod 5, a connecting block 6, an open knob 7, a spring 8, a lower comprehensive cover 9, a U-nail top ring 10, an upper comprehensive cover 11, a thread locking ring 12, a ring cutter 13, a U-nail spacer ring 14, and a ring cutter washer 15. The handle 1 is drivingly connected to the push rod 5 and the connecting block 6 through the small link 3 and the pin 4. The open knob 7 is sleeved on the connecting block 6. The plug 2 is arranged at a lower end of the push rod 5. The upper comprehensive cover 11 and the lower comprehensive cover 9 are connected directly or through a connecting cover. The spring 8 is arranged in an inner cavity of the connecting block 6. The U-nail top ring 10 is wholly or partially arranged in the lower comprehensive cover 9, or wholly or partially arranged in the connecting cover, or partially arranged in the lower comprehensive cover 9 and the other part arranged in the connecting cover. The thread locking ring 12 is locked on an outer periphery of the upper comprehensive cover 11 and/or the lower comprehensive cover 9 and/or the connecting cover. The ring cutter 13 is arranged on an outer periphery of the U-nail top ring 10 and the guide block. The U-nail spacer ring 14 is fixed at a top end of a U-nail groove for a U-nail, when being stapled, preferentially passing through the U-nail spacer ring 14 so that one or more sides of the U-nail does not directly contact a prepuce when the U-nail is stapled into the prepuce issue. The principles of other related driving components, actuating components and ringing components are the same as those in the previous embodiment, which will be not described herein.

The valgus prepuce circumcision stapler with an indication device includes an upper comprehensive cover, a lower comprehensive cover and/or a connecting cover, a prepuce fixing hoop and a U-nail device; and further includes an indication device. The upper comprehensive cover and the lower comprehensive cover are vertically assembled to and cooperating with each other. Or, a lower part of the upper comprehensive cover is connected to the connecting cover and a lower part of the connecting cover is connected to the lower comprehensive cover. The prepuce fixing hoop is embedded in the upper comprehensive cover. Or, the prepuce fixing hoop is integrated with the upper comprehensive cover. The U-nail device is wholly or partially arranged in the lower comprehensive cover. Or, the U-nail device is wholly or partially arranged in the connecting cover. Or, the U-nail device is partially arranged in the lower comprehensive cover and the other part is arranged in the connecting cover. The indication device is arranged on the upper comprehensive cover, and/or the lower comprehensive cover, and/or the prepuce fixing hoop, and/or the U-nail device.

Also included is a locking ring which is used for locking the upper comprehensive cover, the connecting cover and/or the lower comprehensive cover and is provided with the indication device. The indication device is a loop of protrusions or recesses. Preferably, the concave or convex degree of the protrusions or recesses allows that the protrusions or recesses can be contacted and perceived by an operator when the prepuce covers or does not cover the protrusions or recesses. The U-nail device includes a U-nail top ring and a thimble guide block for accommodating suturing U-nails, and/or guiding the movement of the suturing U-nails, and/or positioning and/or guiding a thimble of the U-nail top ring. The U-nail top ring is used for pushing the U-nails therein out in cooperation with the thimble top block. The indication device is arranged on the thimble guide block. The thimble guide block is provided with a plurality of thimble guide grooves which are annularly arranged on the annular thimble guide block and are capable of accommodating the suturing U-nails.

The protrusions or recesses of the indication device are arranged to be in one-to-one correspondence with the thimble guide grooves and/or intervals among the thimble guide grooves and/or U-nails in the thimble guide grooves. Or, intervals among the protrusions or recesses of the indication device are in one-to-one correspondence with the thimble guide grooves and/or the intervals among the thimble guide grooves and/or U-nails in the thimble guide grooves. The protrusions or recesses of the indication device are arranged such that the width between the two protrusions or recesses is equal to or slightly smaller than the width of the thimble guide groove, and/or the width of the U-nail in the thimble guide groove, and/or the width of the interval between the thimble guide grooves.

The indication device is protrusions or recesses which are prefabricated on the upper comprehensive cover, and/or the lower comprehensive cover, and/or the connecting cover, and/or the prepuce fixing hoop, and/or the U-nail device, and/or the locking ring. And/or, the indication device is an indication label which is adhered to the upper comprehensive cover, and/or the lower comprehensive cover, and/or the connecting cover, and/or the prepuce fixing hoop, and/or the U-nail device, and/or the locking ring and has protrusions or recesses. And/or, the indication device is a prefabricated indication ring or indication strip which sleeves the upper comprehensive cover, and/or the lower comprehensive cover, and/or the connecting cover, and/or the prepuce fixing hoop, and/or the U-nail device, and/or the locking ring and has protrusions or recesses. And/or, the indication device is protrusions or recesses which are prefabricated on the periphery of the upper end and/or the periphery of the lower end of the prepuce fixing hoop, and/or an indication label which is adhered to the periphery of the upper end and the periphery of the lower end of the prepuce fixing hoop and has protrusions or recesses, and/or prefabricated indication ring or strip which sleeves the periphery of the upper end and/or the periphery of the lower end of the prepuce fixing hoop and has protrusions or recesses. And/or, the indication device is protrusions or recesses which are prefabricated on the periphery of the upper end and/or the periphery of the lower end of the thimble guide block, and/or an indication label which is adhered to the periphery of the upper end and the periphery of the lower end of the thimble guide block and has protrusions or recesses, and/or a prefabricated indication ring or strip which sleeves the periphery of the upper end and/or the periphery of the lower end of the thimble guide block and has protrusions or recesses. And/or, the indication device is graduations prefabricated on the periphery of the upper end and/or the periphery of the lower end of the prepuce fixing hoop, and/or a graduation label adhered to the periphery of the upper end and the periphery of the lower end of the prepuce fixing hoop, and/or a prefabricated graduation ring or graduation strip which sleeves the periphery of the upper end and the periphery of the lower end of the prepuce fixing hoop, and/or a blood vessel docking groove which is prefabricated on the periphery of the upper end and the periphery of the lower end of the prepuce fixing hoop. And/or, the indication device is graduations prefabricated on the periphery of the upper end and/or the periphery of the lower end of the U-nail device, and/or a graduation label adhered to the periphery of the upper end and the periphery of the lower end of the U-nail device, and/or a prefabricated graduation ring or graduation strip which sleeves the periphery of the upper end and the periphery of the lower end of the U-nail device, and/or a blood vessel docking groove which is prefabricated on the periphery of the upper end and the periphery of the lower end of the U-nail device.

The protrusions are convex blocks and/or convex points and/or convex strips. The recesses are grooves and/or concave points and/or concave strips. Both the left side surface and the right side surface and/or both the upper end surface and the lower end surface of the convex block have one or a plurality of oblique surface. And/or, one or two of the convex points constitute a group and are circular convex points. And/or one or two of the convex strips constitute a group, and both the left side surface and the right side surface of each convex strip have one or a plurality of oblique surfaces. And/or, both the left side surface and the right side surface and/or both the upper end surface and the lower end surface of the groove have one or a plurality of oblique surfaces. And/or, one or two of the concave points constitute a group and are circular concave points. And/or, one or two of the concave strips constitute a group, and both the left side surface and the right side surface of each concave strip have one or a plurality of oblique surfaces. And/or, the convex strip is a vertical convex strip, and the upper end and the lower end of the convex strip are curved. And/or, the concave strip is a vertical concave strip, and the upper end and the lower end of the convex strip are curved.

The indication device is arranged at one end, close to the upper comprehensive cover, of the outer surface of the lower comprehensive cover. And/or, the indication device is arranged at one end, close to the connecting cover, of the outer surface of the lower comprehensive cover. And/or, the indication device is arranged at one end, close to the lower comprehensive cover or the connecting cover, of the outer surface of the upper comprehensive cover. And/or, the indication device is arranged at the upper end of the outer surface of the upper comprehensive cover. And/or, the indication device is arranged at one end, close to the upper comprehensive cover, of the outer surface of the connecting cover. And/or, the indication device is arranged at one end, close to the lower comprehensive cover, of the outer surface of the connecting cover. And/or, the indication device is arranged at the upper end of the outer surface of the prepuce fixing hoop. And/or, the indication device is arranged at the lower end of the outer surface of the prepuce fixing hoop. And/or, the indication device is arranged at the upper end of the outer surface of the U-nail device. And/or, the indication device is arranged at the lower end of the outer surface of the U-nail device. And/or, the indication device is annularly arranged on the periphery of the upper part/the lower part of the thimble guide block.

The above description of the present invention has been given by way of example with reference to the accompanying drawings. It is obvious that the specific implementation of the present invention is not limited to the above modes, and various improvements made using the method concepts and technical solutions of the present invention or directly applied to other occasions, are within the scope of the present invention.

Refer to the operation-visible circumcision stapler.

The present invention is described in detail below according to the accompanying drawings, which is a preferred embodiment of various embodiments of the present invention.

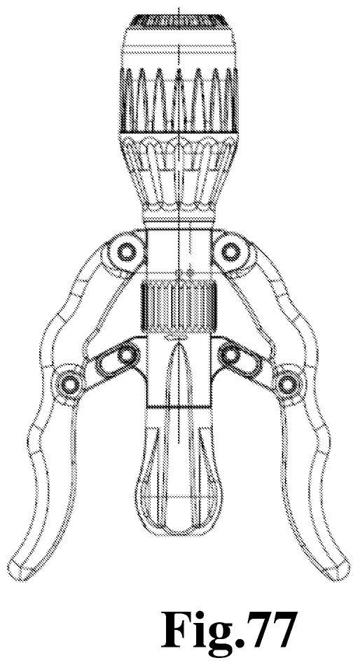

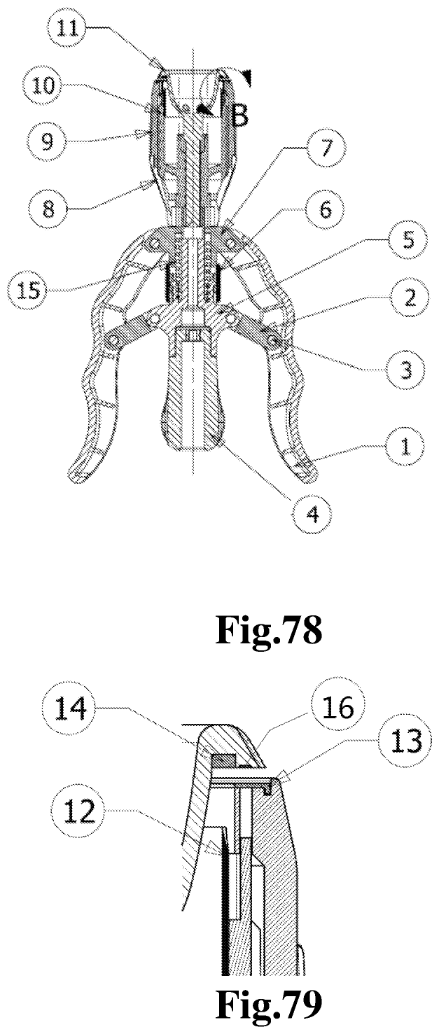

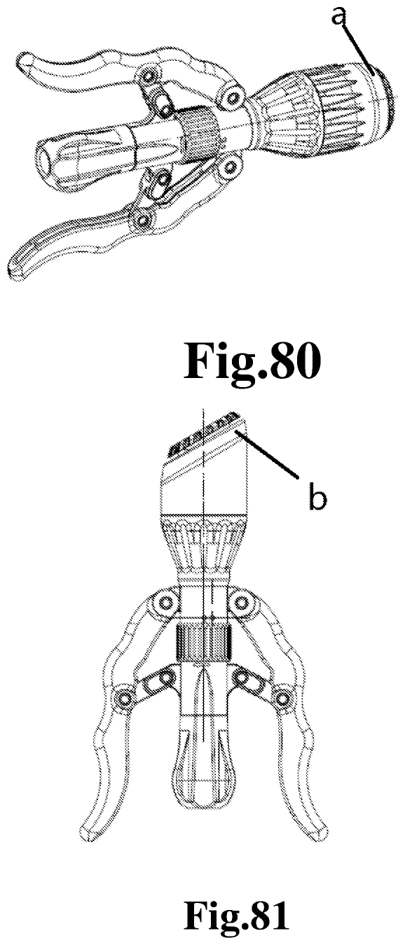

FIG. 77 is an overall view of a flat-head circumcision stapler of the present invention and FIGS. 78 and 79 are structural views of the flat-head circumcision stapler of the present invention. The flat-head circumcision stapler comprises a handle 1, a connecting rod 2, a rivet 3, a knob 4, a sheathed core 5, an opening knob 6, a sheathed handle 7, a lower sleeve 8, a comprehensive cover 9, a thimble 10, a circumcision cover 11, a circumcision knife 12, a first washer 13, a third washer 14, a spring 15 and a second washer 16. The circumcision cover 11 is inserted from the top end of the comprehensive cover 9. The ring-shaped lower end of the upper portion of the circumcision cover 11 cooperates with the top end of the comprehensive cover 9 for clamping the prepuce. The lower end of the comprehensive cover 9 is connected with the lower sleeve 8. The U-nail top ring is embedded into the comprehensive cover 9 and the lower sleeve 8 and is provided with the thimble 10 at the top. The lower end of the lower sleeve 8 is connected to the sheathed handle 7. The lower end of the sheathed handle 7 is connected to the sheathed core 5. The knob 4 is arranged, as a plug, at the tail end of the sheathed core 5. The circumcision knife is arranged in the U-nail top ring. The first washer 13, the second washer 16 and the third washer 14 are arranged at the top end of the U-nail groove, and an outer ring and an inner ring of a cutting surface of the circumcision cover, respectively. The handle 1, the connecting rod 2 and the rivet 3 constitute a driving component and are connected to the sheathed core 5 and the sheathed handle 7 and used to drive the U-nail top ring and/or the circumcision knife 12, thereby completing circumcision and suturing. The opening knob 6 sleeves the sheathed core 5 and is used to lock and unlock the driving component. FIG. 80 is a stereoscopic view of the flat-head circumcision stapler of the present invention; and the position indicated by a is a position where the U-nail groove is observed for positioning during a surgery.

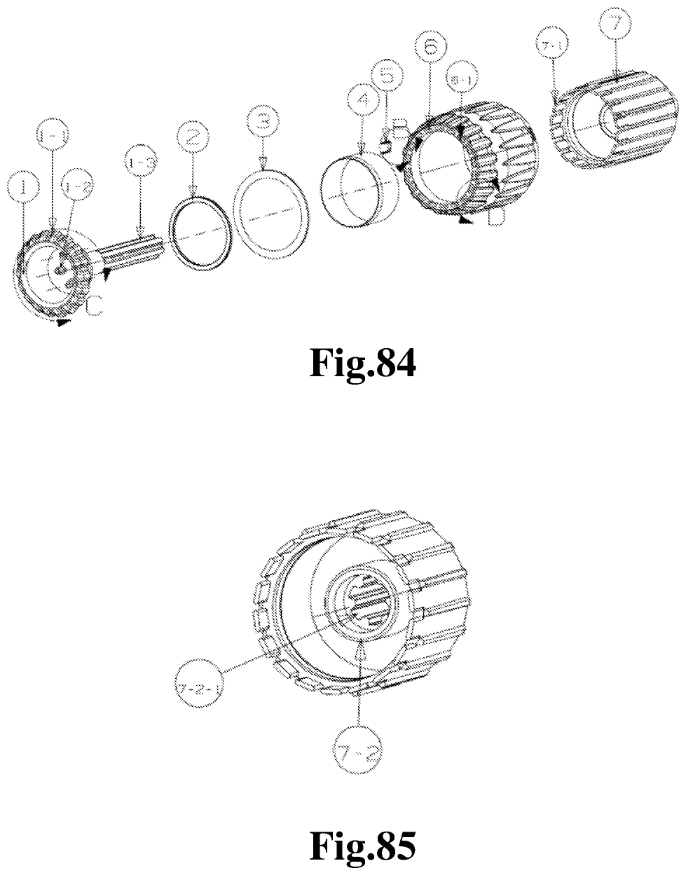

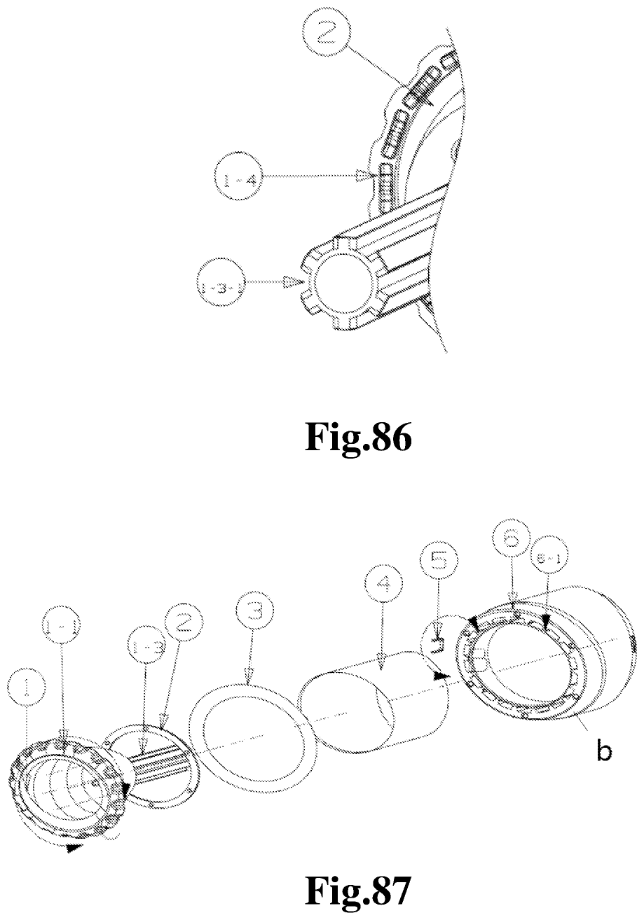

FIGS. 84-86 are a diagram and partial enlarged views of the position of a nail cabin of a flat-head built-in circumcision stapler. In the figures, 1 represents a circumcision cover, 1-1 blood vessel corresponding grooves, 1-2 a negative pressure hole, 1-3 a positioning rod, 1-3-1 a positioning groove, 1-4 a U-nail seat, 2 a circumcision knife washer, 3 a U-nail backing ring, 4 a circumcision knife, 5 a U-nail, 6 a comprehensive cover, 6-1 a U-nail groove, 7 a U-nail top ring, 7-1 a thimble, 7-2 a positioning hole, and 7-2-1 a positioning groove. The operation-visible circumcision stapler comprises the comprehensive cover 6, the circumcision cover 1, the U-nail top ring 7 and the circumcision knife 4. The circumcision cover 1 has a lower end inserted into the comprehensive cover 6 and an upper end supported at the top end of the comprehensive cover 6 for cooperating with the comprehensive cover 6 to clamp prepuce. The comprehensive cover 6 is transparent and a plurality of U-nail grooves 6-1 which is used for accommodating U-nails and constitutes a ring is arranged in the comprehensive cover 6. There are intervals among the U-nail grooves 6-1, the inner surfaces and/or outer surfaces of the U-nail grooves 6-1 are made of a transparent material, and the intervals are made of a semi-transparent material. The U-nail top ring 7 is embedded into the comprehensive cover 6, thimbles 7-1 in one-to-one correspondence with the U-nail grooves 6-1 are arranged at the top end of the U-nail top ring, and the thimble 7-1 of the U-nail top ring 7 or the entire U-nail top ring 7 is transparent. The circumcision knife 4 is embedded into the U-nail top ring 7.

Blood vessel corresponding grooves 1-1 are annularly arranged at the upper end of the circumcision cover 1 and are in one-to-one correspondence with the U-nail grooves 6-1. The positioning rod 1-3 is arranged at the lower end of the circumcision cover 1, the positioning hole 7-2 is formed in the U-nail top ring 7, and the positioning rod 1-3 is configured to be inserted in and be positioned in the positioning hole 7-2, so that the blood vessel corresponding grooves 1-1 on the circumcision cover 1 are in one-to-one correspondence with the U-nail grooves 6-1.

A ring of blood vessel docking grooves/graduations is annularly arranged on the outer surface of the comprehensive cover 6, and the blood vessel docking grooves/graduations are in one-to-one correspondence with the U-nail grooves 6-1 and/or blood vessel corresponding grooves 1-1. With reference to the drawings, position relationships and connection relationships of other relevant driving components, actuation components and circumcision components are clear, and the details will not be repeated here.

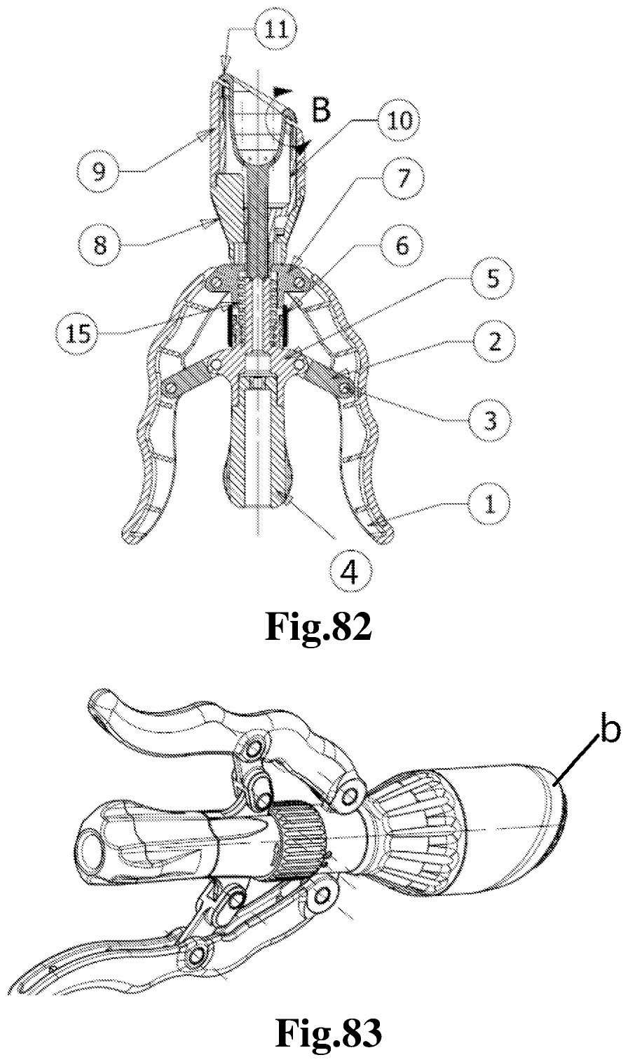

FIG. 81 is an overall view of an oblique-head circumcision stapler of the present invention; and the position indicated by b in this figure is a position where the U-nail groove is observed for positioning during a surgery. FIG. 82 is a structural view of the oblique-head circumcision stapler of the present invention, which comprises a handle 1, a connecting rod 2, a rivet 3, a knob 4, a sheathed core 5, an opening knob 6, a sheathed handle 7, a lower sleeve 8, a comprehensive cover 9, a thimble 10, a circumcision cover 11, a circumcision knife 12, a first washer 13, a third washer 14, a spring 15 and a second washer 16. With reference to the drawings, position relationships and connection relationships of other relevant driving components, actuation components and circumcision components are clear, and the details will not be repeated here. FIG. 83 is a stereoscopic view of the oblique-head circumcision stapler of the present invention; and the position indicated by b in this figure is a position where the U-nail groove is observed for positioning during surgery.

FIG. 87 is a diagram of the position of a nail cabin of a flat-head built-in circumcision stapler. In the figure, 1 represents a circumcision cover, 1-1 blood vessel corresponding grooves, 1-2 a negative pressure hole, 1-3 a positioning rod, 1-3-1 a positioning groove, 1-4 a U-nail seat, 2 a circumcision knife washer, 3 a U-nail backing ring, 4 a circumcision knife, 5 a U-nail, 6 comprehensive cover and 6-1 a U-nail groove. With reference to the drawings, position relationships and connection relationships of other relevant driving components, actuation components and circumcision components are clear, and the details will not be repeated here.

The above description of the present invention has been given by way of example with reference to the accompanying drawings. It is obvious that the specific implementation of the present invention is not limited to the above modes, and various improvements made using the method concepts and technical solutions of the present invention or directly applied to other occasions, are within the scope of the present invention.

1. A blood vessel docking apparatus for a built-in circumcision device, comprising a circumcision cover and a comprehensive cover, and further comprising blood vessel corresponding grooves and/or blood vessel docking grooves, wherein
the circumcision cover and the comprehensive cover are configured to be assembled or connected with each other;
a plurality of blood vessel corresponding grooves is disposed along a periphery of a front end of the circumcision cover; and/or
a plurality of blood vessel docking grooves is disposed along a periphery of a front end of the comprehensive cover.

2. The blood vessel docking apparatus for a built-in circumcision device according to claim 1, wherein a top end of the comprehensive cover is provided with an annular outer wall, the blood vessel docking grooves are disposed on an outer surface of the annular outer wall, front ends of the blood vessel docking grooves are flush with a top end of the annular outer wall, or the front ends of the blood vessel docking grooves are higher than the top end of the annular outer wall, or the front ends of the blood vessel docking grooves are lower than the top end of the annular outer wall.

3. The blood vessel docking apparatus for a built-in circumcision device according to claim 2, wherein the comprehensive cover is provided with a plurality of U-nail grooves for receiving U-nails and nail outlets of the U-nail grooves are disposed on or close to the top end of the comprehensive cover;
wherein the plurality of blood vessel corresponding grooves corresponds to the blood vessel docking grooves, and/or the U-nail grooves, and/or the U-nails in the U-nail grooves one by one; and/or, the plurality of blood vessel docking grooves corresponds to the blood vessel corresponding grooves, and/or the U-nail grooves, and/or the U-nails in the U-nail grooves one by one; and/or the width of the plurality of blood vessel corresponding grooves corresponds to the width of the blood vessel docking grooves, and/or the width of the U-nail grooves, and/or the width of the U-nails in the U-nail grooves; and/or, the width of the plurality of blood vessel docking grooves corresponds to the width of the blood vessel corresponding grooves, and/or the width of the U-nail grooves, and/or the width of the U-nails in the U-nail grooves.

4. A blood vessel docking apparatus for a valgus circumcision device, comprising an inner ring, an inner comprehensive cover and blood vessel docking grooves, wherein
the inner ring and the inner comprehensive cover are configured to be assembled or connected with each other; and
a plurality of blood vessel docking grooves is disposed along a periphery of a back end of the inner ring.

5. The blood vessel docking apparatus for a valgus circumcision device according to claim 4, wherein the inner comprehensive cover is provided with a plurality of U-nail grooves for receiving U-nails and the U-nail grooves are disposed along a periphery of a front end of the inner comprehensive cover;
wherein positions of the plurality of blood vessel docking grooves correspond to the positions of the U-nail grooves one by one, and/or the positions of the U-nails in the U-nail grooves; and/or the width of the plurality of blood vessel docking grooves corresponds to the width of the U-nail grooves, and/or the width of the U-nails in the U-nail grooves.

6. The blood vessel docking apparatus for a valgus circumcision device according to claim 4, further comprising a U-nail top ring, configured to be assembled and matched with the inner comprehensive cover to push the U-nail out of the U-nail groove;
further comprising an upper cover and a lower cover, wherein the upper cover and the lower are assembled and connected with each other, the inner ring is arranged in the upper cover, and all or part of the inner comprehensive cover is disposed in the lower cover;
further comprising a comprehensive cover, wherein a front end of the comprehensive cover is connected to a back end of the lower cover, and all or part of the U-nail top ring is disposed in the comprehensive cover.

7. A built-in graduated circumcision device comprising a comprehensive cover, a circumcision cover and a graduation device, wherein
the comprehensive cover and circumcision cover are assembled to and cooperate with each other; and
the graduation device is arranged on the comprehensive cover.

8. The built-in graduated circumcision device of claim 7, wherein a plurality of U-nail grooves which constitutes a ring is arranged in the comprehensive cover;
wherein graduation marks on the graduation device are arranged to be in one-to-one correspondence with the U-nail grooves and/or intervals among the U-nail grooves and/or U-nails in the U-nail grooves.

9. The built-in graduated circumcision device of claim 7, wherein a ring of blood vessel docking grooves are annularly arranged on the outer surface of the comprehensive cover, and the graduation marks on the graduation device are arranged to be in one-to-one correspondence with the blood vessel docking grooves and/or intervals among the blood vessel docking grooves;
wherein a ring of blood vessel corresponding grooves are annularly arranged on an outer surface of the circumcision cover, and the graduation marks on the graduation device are arranged to be in one-to-one correspondence with the blood vessel corresponding grooves and/or the intervals among the blood vessel corresponding grooves.

10. A valgus graduated circumcision device comprising an upper cover and/or a lower cover, and/or a locking ring, and further comprising a graduation device, wherein:
the upper cover and the lower cover are assembled to and cooperate with each other;
the locking ring is used for locking the upper cover and the lower cover; and
the graduation device is arranged on the upper cover, and/or the lower cover, and/or the locking ring.

11. The valgus graduated circumcision device of claim 10, further comprising an inner comprehensive cover arranged in the lower cover, wherein a plurality of U-nail grooves are annularly arranged on a periphery of the inner comprehensive cover and constitute a ring;
wherein graduation marks on the graduation device are arranged to be in one-to-one correspondence with the U-nail grooves and/or intervals among the U-nail grooves and/or U-nails in the U-nail grooves.

12. A built-in prepuce circumcision stapler with an indication device, comprising a comprehensive cover, a prepuce circumcision cover and the indication device, wherein,
the comprehensive cover and the prepuce circumcision cover are assembled to and cooperate with each other;
the indication device is arranged on the comprehensive cover.

13. The built-in prepuce cutting stapler with the indication device according to claim 12, wherein the indication device is a ring of protrusions or recesses which are annularly arranged on the periphery of one end, close to the prepuce circumcision cover, of the comprehensive cover; preferably, the concave or convex degree of the protrusions or recesses allows that the protrusions or recesses can be contacted and perceived by an operator when the prepuce covers or does not cover the protrusions or recesses.

14. The built-in prepuce circumcision stapler with the indication device according to claim 12, wherein a plurality of U-nail grooves which serves to accommodate U-nails and constitutes a ring is arranged in the comprehensive cover;
wherein the protrusions or recesses of the indication device are arranged to be in one-to-one correspondence with the U-nail grooves and/or intervals among the U-nail grooves and/or the U-nails in the U-nail grooves; or, intervals among the protrusions or recesses of the indication device are in one-to-one correspondence with the U-nail grooves and/or the intervals among the U-nail grooves and/or the U-nails in the U-nail grooves.

15. The built-in prepuce circumcision stapler with the indication device according to claim 12, wherein a ring of blood vessel docking grooves are annularly arranged on the outer surface of the comprehensive cover, and the protrusions or recesses on the indication device are arranged to be in one-to-one correspondence with the blood vessel docking grooves and/or intervals among the blood vessel docking grooves; and/or a ring of blood vessel corresponding grooves are annularly arranged on the outer surface of the prepuce circumcision cover, and the protrusions or recesses on the indication device are arranged to be in one-to-one correspondence with the blood vessel corresponding grooves and/or intervals among the blood vessel corresponding grooves.

16. A valgus prepuce circumcision stapler with an indication device, comprising an upper comprehensive cover, a lower comprehensive cover and/or a connecting cover, a prepuce fixing hoop, a U-nail device and the indication device, wherein,
the upper comprehensive cover and the lower comprehensive cover are assembled to and cooperate with each other; or, the lower part of the upper comprehensive cover is connected to the connecting cover, and the lower part of the connecting cover is connected to the lower comprehensive cover;
the prepuce fixing hoop is arranged in the upper comprehensive cover, or the prepuce fixing hoop and the upper comprehensive cover are of an integral structure;
the U-nail device is fully or partially arranged in the lower comprehensive cover; or the U-nail device is fully or partially arranged in the connecting cover; or, a part of the U-nail device is arranged in the lower comprehensive cover, and the other part of the U-nail device is arranged in the connecting cover;
the indication device is arranged on the upper comprehensive, and/or the lower comprehensive cover, and/or the connecting cover, and/or the prepuce fixing hoop, and/or the U-nail device.

17. The valgus prepuce circumcision stapler with the indication device according to claim 16, further comprising a locking ring which is used for locking the upper comprehensive cover, and/or the connecting cover, and/or the lower comprehensive cover and is provided with the indication device.

18. The valgus prepuce circumcision stapler with the indication device according to claim 16, wherein the indication device is a ring of protrusions or recesses which are annularly arranged; preferably, the concave or convex degree of the protrusions or recesses allows that the protrusions or recesses can be contacted and perceived by an operator when the prepuce covers or does not cover the protrusions or recesses;
wherein the U-nail device comprises a U-nail top ring and a thimble guide block; the thimble guide block is used for accommodating suturing U-nails, and/or guiding the movement of the suturing U-nails, and/or positioning and/or guiding a thimble of the U-nail top ring; the U-nail top ring is used for pushing the U-nails in the thimble top block out in cooperation with the thimble top block; the indication device is arranged on the thimble guide block;
wherein the thimble guide block is provided with a plurality of thimble guide grooves which are annularly arranged on the annular thimble guide block and are capable of accommodating the suturing U-nails;
wherein the protrusions or recesses of the indication device are arranged to be in one-to-one correspondence with the thimble guide grooves and/or intervals among the thimble guide grooves and/or U-nails in the thimble guide grooves; or, intervals among the protrusions or recesses of the indication device are in one-to-one correspondence with the thimble guide grooves and/or the intervals among the thimble guide grooves and/or U-nails in the thimble guide grooves.

19. An operation-visible circumcision stapler, comprising a comprehensive cover and a circumcision cover, wherein
the comprehensive cover and the circumcision cover are assembled to and cooperate with each other; and
an upper portion, close to the circumcision cover, of the comprehensive cover or the entire comprehensive cover is transparent or semi-transparent.

20. The operation-visible circumcision stapler according to claim 19, wherein a plurality of blood vessel corresponding grooves is annularly arranged at the upper end of the circumcision cover and is in one-to-one correspondence with the U-nail grooves;
wherein a ring of blood vessel docking grooves/graduations is annularly arranged on the outer surface of the comprehensive cover, and the blood vessel docking grooves/graduations are in one-to-one correspondence with the U-nail grooves and/or blood vessel corresponding grooves.
